(19) United States
(12) Patent Application Publication
(10) Pub. No.: US 2008/0176827 A1
(43) Pub. Date: Jul. 24, 2008

(54) New Pyridine Analogues VII 543

(75) Inventors: Thomas Antonsson, Molndal (SE); Peter Bach, Molndal (SE); Kay Brickmann, Molndal (SE); Ruth Bylund, Molndal (SE); Fabrizio Giordanetto, Molndal (SE); Johan Johansson, Molndal (SE); Fredrik Zetterberg, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/972,654

(22) Filed: Jan. 11, 2008

Related U.S. Application Data
(60) Provisional application No. 60/884,660, filed on Jan. 12, 2007.

(51) Int. Cl. A61K 31/397; C07D 211/00; A61K 31/445; C07D 401/04; A61P 9/00; A61K 31/4439; A61K 31/4427

(52) U.S. Cl. 514/210.2; 546/193; 514/318; 514/343; 546/276.4; 546/268.1

(57) ABSTRACT

The present invention relates to certain new pyridin analogues of Formula (I)

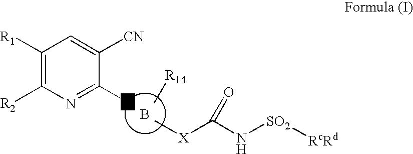

to processes for preparing such compounds, to their utility as P2Y₁₂ inhibitors and as anti-trombotic agents etc, their use as medicaments in cardiovascular diseases as well as pharmaceutical compositions containing them.

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation. WO 2002/098856 and WO 2004/052366 describe piperazino-carbonylmethylaminocarbonyl-naphtyl or -quinolyl derivatives as ADP receptor antagonist.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31 (2): 195-204 JJJ van Giezen & R G Humphries. Preclinical and clinical studies with selective reversible direct P2Y₁₂ antagonists. WO 2005/000281 describes a serie of pyrazolidine-3,5-dione derivatives and WO 2006/1147742 describes a serie of phenyl-pyrimidine derivatives which both series have been described as P2Y12 antagonists for the potential treatment of thrombosis. WO 2006/073361 discloses some P2Y12 antagonists for the potential treatment of thrombosis.

It is an object of the present invention to provide improved potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below (See p. 49-50). Examples of such beneficial properties are high potency, high selectivity, and an advantageous therapeutic window.

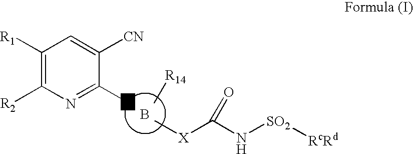

### DETAILED DESCRIPTION OF THE INVENTION

According to the present invention there is provided a novel compound of formula (I) or a pharmaceutically acceptable salt thereof:

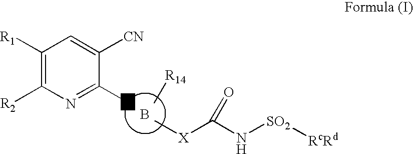

wherein

R₁ represents R₆OC(O);

R₂ represents methyl, ethyl or methylamino;

R₆ represents ethyl or halogenated ethyl;

R₁₄ represents H, (C₁-C₁₂)alkyl optionally interrupted by oxygen and/or optionally substituted by one or more of OH, COOH and COOR³; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl;

R^{c} represents methylene (—CH₂—) or ethylene (—CH₂—CH₂—);

R^{d} represents phenyl, wherein the phenyl group optionally is;
substituted by one or more fluorine atom(s), and/or
substituted by one or more chlorine atom(s) only in one or more of the 2,4,5,6-positions of the phenyl ring, and/or
mono-, tri-, tetra- or penta-substituted by methyl group(s), and/or
substituted by one or more methoxy group(s) only in one or more of the 2,3,5,6-positions of the phenyl ring;
X represents a single bond or methylene (—CH₂—), wherein the methylene group may optionally be substituted with (C₁-C₆) alkyl; further X may represent a group (—CH₂—)n wherein n=2-6, which optionally is unsaturated and/or substituted by one or more substituent chosen among halogen, hydroxyl or (C₁-C₆)alkyl;
B is a ring/ring system comprising a nitrogen which nitrogen is connected to the pyridine-ring (according to formula I) and further the B-ring/ring system is connected to X in another of its positions. The substituent R₁₄ is connected to the B ring/ring system in such a way that no quarternary ammonium compounds are formed (by this connection). The B ring/ring system is further chosen from the group consisting of 3-azetidin-1-ylene, 3-pyrrolidine-1-ylene and 4-piperidine-1-ylene; and

wherein the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate ethyl 6-(3-{[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.
   Preferred values of each variable group or specific embodiments of variable groups or terms are as follows. Such values or embodiments may be used where appropriate with any of the values, definitions, claims, aspects, embodiments or embodiments of the invention defined hereinbefore or hereinafter. In particular, each may be used as an individual limitation on the broadest definition of formula (I).
   For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention. It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, CN, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkyl” includes both linear or branched chain groups, optionally substituted by one or more halogens (F, Cl, Br, I) or mixed halogen atoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, optionally substituted by one or more halogens (F, Cl, Br, I) or mixed halogen atoms.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R^{c} when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₆ represents ethyl or 2,2,2-trifluoroethyl.
In another embodiment of the invention R₆ represents halogenated ethyl.
In a further embodiment of the invention R₆ represents fluorinated ethyl.
In an even further embodiment of the invention R₆ represents ethyl.
In one embodiment of the invention R₂ is methyl or ethyl.
In another embodiment of the invention R₂ is methyl or methylamino.
In a further embodiment of the invention R₂ is ethyl or methylamino.
In one embodiment of the invention R₁₄ is carboxy(C₁-C₁₂)alkyl.

In an even further embodiment R₁₄ is (C₁-C₁₂)alkyl substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl;

Other embodiments for R₁₄ include hydrogen and (C₁-C₁₂)alkyl.

Further embodiments for R₁₄ include hydrogen and (C₁-C₆)alkyl.

In a further embodiment R₁₄ is (C₁-C₁₂)alkyl substituted by COOR^{e}, wherein R^{e} represents aryl.

Another embodiment of R₁₄ is 3-(benzyloxy)-3-oxopropyl.

Even further embodiments for R₁₄ include hydrogen and methyl.

In one embodiment R^{c} represents methylene (—CH₂—).

In another embodiment R^{c} represents ethylene (—CH₂—CH₂—).

Embodiments for R^{d} include phenyl substituted with a fluorine atom at anyone of the positions of the phenyl ring.

Other embodiments for R^{d} include phenyl substituted with a fluorine atom at anyone of the positions of the phenyl ring and one or more chlorine atom(s) only positioned at any of the unoccupied 2,4, 5 or 6-positions of the phenyl ring.

Further embodiments for R^{d} include phenyl substituted with a fluorine atom at anyone of the 2,4, 5 or 6-positions of the phenyl ring and a methyl group at anyone of the other positions of the phenyl ring.

Even further embodiments for R^{d} include phenyl substituted with two or more fluorine atoms at any of the positions of the phenyl ring and optionally a methyl group at anyone of the other positions of the phenyl ring.

In one embodiment of the invention X represents a single bond.

In another embodiment of the invention X represents methylene (—CH₂—)
In yet another embodiment X represents methylene (—CH₂—) substituted with (C₁-C₆)alkyl.

In one embodiment of the invention B represents 3-azetidin-1-ylene or 3-pyrrolidine-1-ylene.

In another embodiment of the invention B represents 3-azetidin-1-ylene or 4-piperidine-1-ylene.

In yet another embodiment of the invention B represents 3-pyrrolidine-1-ylene or 4-piperidine-1-ylene.

A 2nd embodiment of formula I is defined by;

R₁ represents R₆OC(O);

R₂ represents methyl, ethyl or methylamino;

R₆ represents ethyl or fluorinated ethyl;

R₁₄ represents H, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl;

R^{c} represents methylene (—CH₂—) or ethylene (—CH₂—CH₂—).

R^{d} represents phenyl, wherein the phenyl group optionally is;
substituted by one or more fluorine atom(s), and/or
substituted by one or more chlorine atom(s) only in one or more of the 2,4,5,6-positions of the phenyl ring, and/or
mono-, tri-, tetra- or penta-substituted by methyl group(s), and/or
substituted by one or more methoxy group(s) only in one or more of the 2,3,5,6-positions of the phenyl ring;
X represents a single bond or methylene (—CH₂—), wherein the methylene group may optionally be substituted with (C₁-C₆) alkyl; further X may represent a group (—CH₂—)n wherein n=2-6, which optionally is unsaturated and/or substituted by one or more substituent chosen among halogen, hydroxyl or (C₁-C₆)alkyl;
B is a ring/ring system comprising a nitrogen which nitrogen is connected to the pyridine-ring (according to formula I) and further the B-ring/ring system is connected to X in another of its positions. The substituent R₁₄ is connected to the B ring/ring system in such a way that no quarternary ammonium compounds are formed (by this connection). The B ring/ring system is further chosen from the group consisting of 3-azetidin-1-ylene, 3-pyrrolidine-1-ylene and 4-piperidine-1-ylene; and

wherein the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate
ethyl 6-(3-{[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.

A 3rd embodiment of formula I is defined by;

R₁ represents R₆OC(O);

R₂ represents methyl, ethyl or methylamino;

R₆ represents ethyl or fluorinated ethyl;

R₁₄ represents H, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl;

R^{c} represents methylene (—CH₂—) or ethylene (—CH₂—CH₂—).

R^{d} represents phenyl, wherein the phenyl group optionally is;
substituted by one or more fluorine atom(s), and/or
substituted by one or more chlorine atom(s) only in one or more of the 2,4,5,6-positions of the phenyl ring, and/or
mono-, tri-, tetra- or penta-substituted by methyl group(s), and/or
substituted by one or more methoxy group(s) only in one or more of the 2,3,5,6-positions of the phenyl ring;
X represents a single bond or methylene (—CH₂—);
B is a ring/ring system comprising a nitrogen which nitrogen is connected to the pyridine-ring (according to formula I) and further the B-ring/ring system is connected to X in another of its positions. The substituent R₁₄ is connected to the B ring/ring system in such a way that no quarternary ammonium compounds are formed (by this connection). The B ring/ring system is further chosen from the group consisting of 3-azetidin-1-ylene, 3-pyrrolidine-1-ylene and 4-piperidine-1-ylene; and

wherein the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[([{2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate
ethyl 6-(3-{[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.

A 4rth embodiment of formula I is defined by;

R₁ represents R₆OC(O);

R₂ represents methyl, ethyl or methylamino;

R₆ represents ethyl or 2,2,2-trifluoroethyl;

R₁₄ represents H, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl;

R^{c} represents methylene (—CH₂—);

R^{d} represents phenyl, wherein the phenyl group optionally is;
substituted by one or more fluorine atom(s), and/or
substituted by one or more chlorine atom(s) only in one or more of the 2,4,5,6-positions of the phenyl ring, and/or
mono-, tri-, tetra- or penta-substituted by methyl group(s), and/or
substituted by one or more methoxy group(s) only in one or more of the 2,3,5,6-positions of the phenyl ring;
X represents a single bond or methylene (—CH₂—);
B is a ring/ring system comprising a nitrogen which nitrogen is connected to the pyridine-ring (according to formula I) and further the B-ring/ring system is connected to X in another of its positions. The substituent R₁₄ is connected to the B ring/ring system in such a way that no quarternary ammonium compounds are formed (by this connection). The B ring/ring system is further chosen from the group consisting of 3-azetidin-1-ylene, 3-pyrrolidine-1-ylene and 4-piperidine-1-ylene; and

wherein the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate ethyl 6-(3-{[(benzylsulfonyl)amino]carbonyl}azetidin-1-yl)-5-cyano-2-methylnicotinate ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.
   In a 5^{th} embodiment of formula (I), formula (I) is defined by;
   R₁ is chosen from the group consisting of ethoxycarbonyl and 2,2,2-trifluoroethoxycarbonyl;
   R₂ is chosen from the group consisting of methyl, ethyl and methylamino;
   R₆ is chosen form the group consisting of ethyl and 2,2,2-trifluoroethyl;
   R₁₄ is chosen form the group consisting of hydrogen, methyl, (3-(benzyloxy)-3-oxopropyl) and 2-carboxyethyl;
   R^{c} is chosen from the group consisting of methylene (—CH₂—) and ethylene(—CH₂—CH₂—);
   R^{d} is chosen from the group consisting of phenyl, 4-fluorophenyl, 2-fluorophenyl, 4-chlorophenyl, 2-fluoro-5-methylphenyl, 3-methoxy-phenyl, 2,6-difluoro-phenyl, 2,4-difluoro-phenyl, 3-methyl-4-fluoro-phenyl, 2 chloro-4-fluoro-phenyl, 2-methyl-5-fluoro-phenyl, 2,3,6-trifluoro-phenyl and 2-fluoro-4-chloro-phenyl;

X is a single bond or methylene (—CH₂—); and

B is chosen form the group consisting of 4-piperidin-1-ylene, 4-(3-methyl)piperidin-1-ylene,
3-[3-(benzyloxy)-3-oxopropyl]-4-piperidin-1-ylene, 3-[2-carboxy-ethyl]-4-piperidin-1-ylene,
3-pyrrolidine-1-ylene and 3-azetidin-1-ylene; and

wherein the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate
ethyl 6-{(3-[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.

In a 6th embodiment of formula (I), formula (I) is defined as being any compound(s) of formula (Ia)-(Ii):

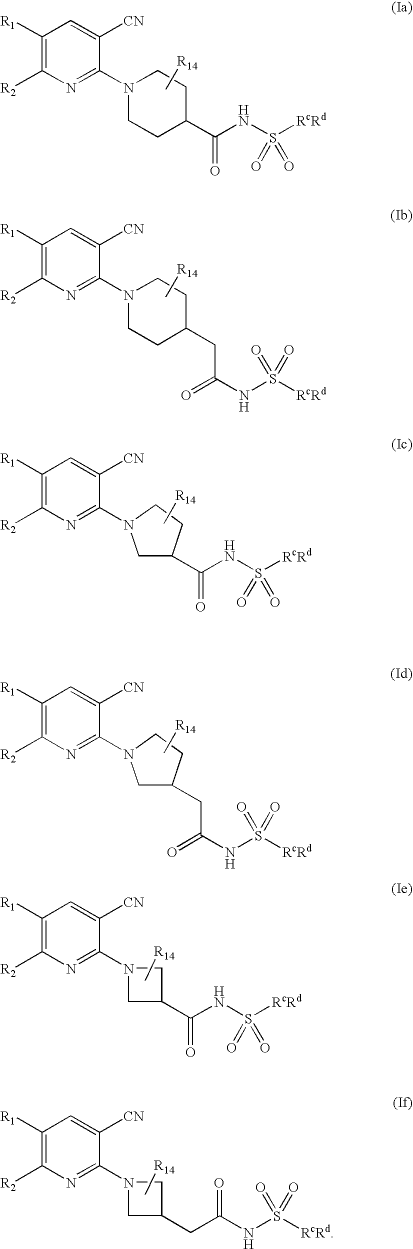

In the above Ia to If the various values of R are as defined above and include the previously mentioned embodiments, but

the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate ethyl 6-(3-{[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.
   In a 7^{th} embodiment formula (I) is defined as being any compound(s) of formula (Iaa)-(Iff);

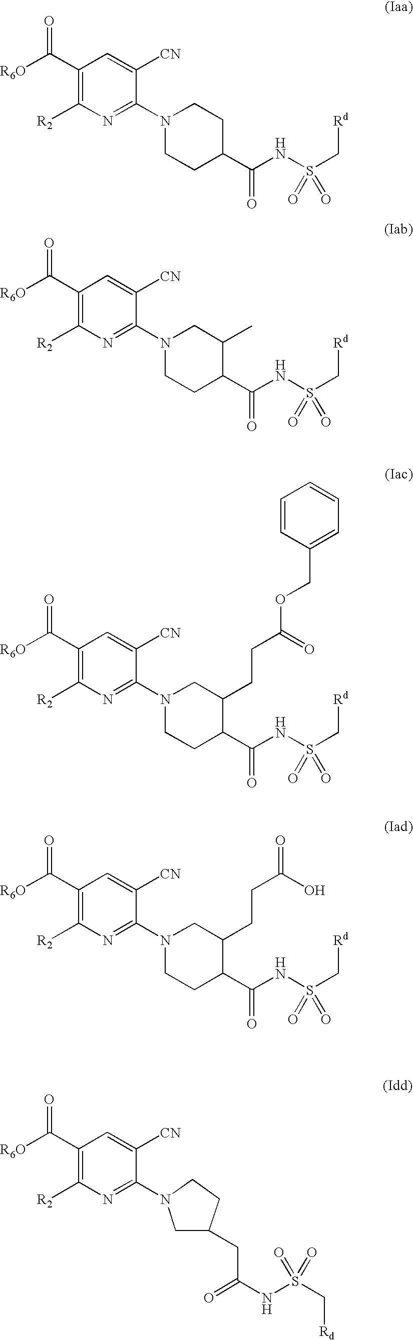

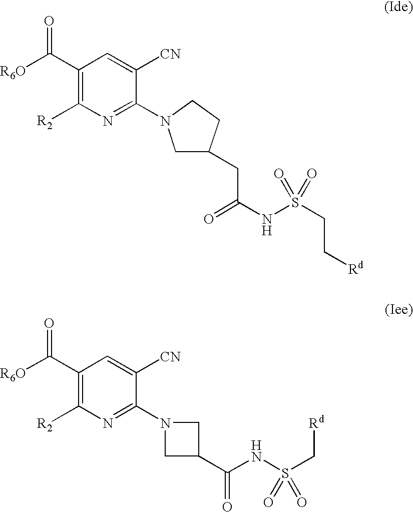

In the above Iaa to Iee the various values of R are as defined above and include the previously mentioned embodiments, but

the following compounds not are included;
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate
ethyl 6-(3-{[(benzylsulfonyl)amino]carbonyl}azetidin-1-yl)-5-cyano-2-methylnicotinate ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[4-({[(3,4-difluorobenzyl)sulfonyl]amino}carbonyl)piperidin-1-yl]-2-methylnicotinate.

Examples of specific compounds according to the invention can be selected from;
ethyl 6-(3-{2-[(benzylsulfonyl)amino]-2-oxoethyl}pyrrolidin-1-yl)-5-cyano-2-methylnicotinate
ethyl 5-cyano-2-methyl-6-[3-(2-oxo-2-{[(2-phenylethyl)sulfonyl]amino}ethyl)pyrrolidin-1-yl]nicotinate
2,2,2-trifluoroethyl 5-cyano-6-(4-{[(4-fluorobenzyl)sulfonyl]carbamoyl}piperidin-1-yl)-2-methylnicotinate
ethyl 5-cyano-6-(3-{[(2-fluoro-5-methylbenzyl)sulfonyl]carbamoyl}azetidin-1-yl)-2-methylnicotinate
ethyl 6-{4-[(benzylsulfonyl)carbamoyl]-3-methylpiperidin-1-yl}-5-cyano-2-methylnicotinate
ethyl 6-{4-[(benzylsulfonyl)carbamoyl]piperidin-1-yl}-5-cyano-2-(methylamino)nicotinate
ethyl 5-cyano-6-(4-{[(2-fluoro-5-methylbenzyl)sulfonyl]carbamoyl}piperidin-1-yl)-2-methylnicotinate
ethyl 5-cyano-6-(4-{[(3-methoxybenzyl)sulfonyl]carbamoyl}piperidin-1-yl)-2-methylnicotinate
ethyl 5-cyano-6-(3-{[(2,6-difluorobenzyl)sulfonyl]carbamoyl}azetidin-1-yl)-2-ethylnicotinate
ethyl 5-cyano-2-ethyl-6-(3-{[(4-fluoro-3-methylbenzyl)sulfonyl]carbamoyl}azetidin-1-yl)nicotinate
ethyl 6-(3-{[(2-chloro-4-fluorobenzyl)sulfonyl]carbamoyl}azetidin-1-yl)-5-cyano-2-ethylnicotinate
ethyl 5-cyano-6-(4-{[(5-fluoro-2-methylbenzyl)sulfonyl]carbamoyl}piperidin-1-yl)-2-methylnicotinate
ethyl 5-cyano-2-methyl-6-(4-{[(2,3,6-trifluorobenzyl)sulfonyl]carbamoyl}piperidin-1-yl)nicotinate
ethyl 5-cyano-6-(4-{[(2,4-difluorobenzyl)sulfonyl]carbamoyl}piperidin-1-yl)-2-methylnicotinate
ethyl 5-cyano-6-(4-{[(2,6-difluorobenzyl)sulfonyl]carbamoyl}piperidin-1-yl)-2-ethylnicotinate
ethyl 6-(4-{[(2-chloro-4-fluorobenzyl)sulfonyl]carbamoyl}piperidin-1-yl)-5-cyano-2-ethylnicotinate
ethyl 5-cyano-2-ethyl-6-(4-{[(2,3,6-trifluorobenzyl)sulfonyl]carbamoyl}piperidin-1-yl)nicotinate
ethyl 5-cyano-6-(4-{[(2,4-difluorobenzyl)sulfonyl]carbamoyl}piperidin-1-yl)-2-ethylnicotinate
ethyl 6-(4-{[(4-chloro-2-fluorobenzyl)sulfonyl]carbamoyl}piperidin-1-yl)-5-cyano-2-ethylnicotinate
ethyl 5-cyano-6-(3-{[(2,4-difluorobenzyl)sulfonyl]carbamoyl}azetidin-1-yl)-2-methylnicotinate
ethyl 5-cyano-6-(3-{[(2,4-difluorobenzyl)sulfonyl]carbamoyl}azetidin-1-yl)-2-ethylnicotinate
ethyl 5-cyano-2-ethyl-6-(3-{[(4-fluorobenzyl)sulfonyl]carbamoyl}azetidin-1-yl)nicotinate
ethyl 6-(3-{[(4-chlorobenzyl)sulfonyl]carbamoyl}azetidin-1-yl)-5-cyano-2-ethylnicotinate
ethyl 5-cyano-2-ethyl-6-(4-{[(4-fluorobenzyl)sulfonyl]carbamoyl}piperidin-1-yl)nicotinate
ethyl 6-(4-{[(4-chlorobenzyl)sulfonyl]carbamoyl}piperidin-1-yl)-5-cyano-2-ethylnicotinate
ethyl 5-cyano-6-(3-{[(2-fluorobenzyl)sulfonyl]carbamoyl}azetidin-1-yl)-2-methylnicotinate
ethyl 5-cyano-6-(4-{[(2-fluorobenzyl)sulfonyl]carbamoyl}piperidin-1-yl)-2-methylnicotinate
ethyl 6-{3-[3-(benzyloxy)-3-oxopropyl]-4-[(benzylsulfonyl)carbamoyl]piperidin-1-yl}-5-cyano-2-methylnicotinate
ethyl 6-(3-[3-(benzyloxy)-3-oxopropyl]-4-{[(2,4-difluorobenzyl)sulfonyl]carbamoyl}piperidin-1-yl)-5-cyano-2-methylnicotinate
3-{4-[(benzylsulfonyl)carbamoyl]-1-[3-cyano-5-(ethoxycarbonyl)-6-methylpyridin-2-yl]piperidin-3-yl}propanoic acid;
   and pharmaceutically acceptable salts thereof.

Processes

The following processes together with the intermediates are provided as a further feature of the present invention.

Compounds of formula (I) may be prepared by the following processes a1-a4;

a1) Compounds of formula (I) in which R₁, X, B, R₁₄, R^{c} and R^{d} are defined as above, R₂ is methyl or ethyl, can be formed by reacting a compound of formula (II), in which R₁, R₂, X, B, and R₁₄ are defined

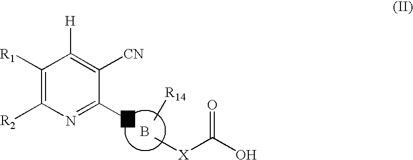

as above with a compound of formula (III) in which R^{c} and R^{d} are defined as above.

H₂NSO₂—R^{c}—R^{d} (III)

The reaction is generally carried out in an inert organic solvent such as dichloromethane at ambient temperature.
The reaction may be carried out using standard conditions or in the presence of TBTU, EDCI, PyBrop or the combination of EDCI and HOBT.
Optionally, the reaction may be carried out in the presence of an organic base such as triethylamine or DIPEA.

a2) Compounds of formula (I) may also be prepared by reacting a compound of formula (IV) in which R₁ is defined as above R₂ is methyl or ethyl and L is a suitable leaving group, such as chloro, bromo, iodo, fluoro, triflate (OTf) or tosylate (OTs),

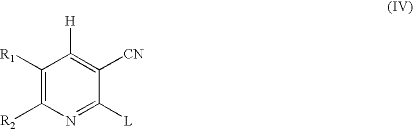

with a compound of the general formula (V) in which B, X, R₁₄, R^{c} and R^{d} are defined as in formula (I).

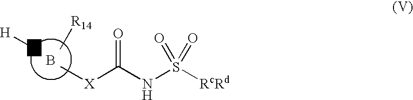

The reaction is generally carried out in an inert solvent such as DMA or DMF.
Optionally, the reaction may be carried out in the presence of an organic base such as triethylamine or DIPEA.
The reaction is generally carried out at elevated temperatures using standard equipment or in a single-node microwave oven.
Generally, using the zwitterion of (V) leads to shorter reaction times than when using the corresponding salt of the B-ring amine, e.g. HCl salt.
For some compounds, it is advantageous to carry out the reaction in ethanol in the presence of an organic base such as triethylamine.

a3) Compounds of formula (I) in which R₁, X, B, R₁₄, R^{c} and R^{d} are defined as above, R₂ is methyl or ethyl, are advantageously prepared by the following steps (a3:1-a3:5),

a3:1) Reacting a compound of the formula R₁CH₂C(O)R₂, with dimethoxy-N,N-dimethylmethaneamine to form a compound of the formula

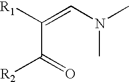

a3:2) This compound is further reacted with a compound of the general formula NC—CH₂C(O)NH₂ to give a compound of the general formula (VI)

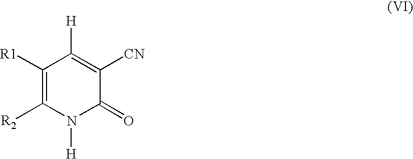

in which R₁ is defined as for formula (I) and R₂ is methyl or ethyl. The reaction is generally performed in an inert solvent such as ethanol. This reaction is performed in the presence of a strong base such as sodium ethoxide. The process is further advantageously performed by washing the final product with an alkaline water solution, e.g. a sodium bicarbonate solution.

a3:3) The compound from a3:2) is reacted with a chlorinating agent such as thionyl chloride to give a compound of formula (IV) wherein L is a chlorine. A further improvement of this reaction is to add dimethylformamide. Advantageously the reaction is performed in an inert solvent such as toluene.

a3:4) Compounds of the general formula (V) in which X, B, R₁₄, R^{c} and R^{d} are defined as above are formed by reacting a compound of formula (VIII) with a compound of formula (III), in which the ring nitrogen is protected, for example by t-butyloxycarbonyl. The reaction is generally carried out in an inert organic solvent such as THF. The reaction is carried out using a coupling reagent such as TBTU. Optionally, the reaction is carried out in the presence of an organic base such as triethylamine or DIPEA. A further improvement of this reaction is to add LiCl. When the product contains a t-butyloxycarbonyl group this group is removed using standard procedures or in the presence of formic acid. In one advantageous embodiment of the process (a3) the product is isolated as a zwitterion by adjusting the pH of the reaction mixture to between approximately 5-9 with ammonia dissolved in water.

a3:5) The product from a3:3 is reacted with the product from a3:4, preferentially the zwitterion, to give a compound of formula (I) in which R₁, X, B, R₁₄, R^{c} and R^{d} are defined as above, R₂ is methyl or ethyl. The reaction is generally carried out in an inert solvent such as ethanol at elevated temperatures. Optionally, the reaction is carried out in the presence of an organic base such as triethylamine.

a(4) Compounds of Formula (I) wherein R₁, X, B, R₁₄, R^{c} and R^{d} are as defined in Formula (I) and R₂ is methylamino (MeNH—) can be formed by reacting a compound of formula (VII),

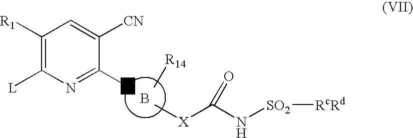

in which R₁, X, B, R₁₄, R^{c} and R^{d} are defined as above and L is a leaving group such as chloro, bromo, iodo, fluoro, triflate (OTf) or tosylate (OTs), with methylamine (CH₃NH₂).

The reaction is generally carried out at elevated temperature using standard equipment or in a single-node microwave oven. The reaction can be carried out in an inert solvent such as THF.
Optionally the reaction may be carried out in the presence of an organic base such as TEA or DIPEA.

The intermediates referred to above may be prepared by, for example, the methods/processes outlined below.

b) The compounds of formula (II) in which R₁, X, B, and R₁₄ are defined as above, R₂ is methyl or ethyl may be prepared by reacting a compound of formula (IV)

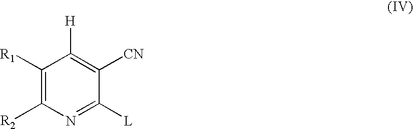

defined as above, with a compound of the general formula (VIII),

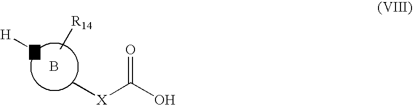

in which X, B and R₁₄ are defined as above.

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

Compounds of the general formula (V) can be formed in one of the processes (c1-c3). The compounds of formula (V) are advantageously isolated as a zwitterion. A ring nitrogen of compounds of formula (V) used in the below steps may be protected by a protective group such as t-butyloxycarbonyl.

c1) Compounds of the general formula (V) in which B, X, R₁₄, R^{c} and R^{d} are defined as above may be formed by reacting a compound of formula (VIII) with a compound of formula (III). The reaction is generally carried out in an inert organic solvent such as dichloromethane at ambient temperature. The reaction may be carried out using standard conditions or in the presence of EDCI or the combination of EDCI and HOBT. Optionally, the reaction may be carried out in the presence of an organic base such as triethylamine or DIPEA.

c2) Compounds of the general formula (V) in which B, X, R₁₄, R^{c} and R^{d} are defined as above may also be formed by reacting a compound of formula (X) with a compound of formula (III), in which the nitrogen in the B-ring is protected, for example by t-butyloxycarbonyl. The reaction is generally carried out in an inert organic solvent such as THF. The reaction may be carried out using standard conditions or in the presence of TBTU. Optionally, the reaction may be carried out in the presence of an organic base such as triethylamine or DIPEA. Advantageously a reagent such as LiCl may be used. When the product contains a t-butyloxycarbonyl this may be removed using standard procedures or in the presence of formic acid. Compounds of formula (V) can be isolated as a zwitterion.

c3) A compound of formula (V) which is protected with t-butoxy carbonyl may be transformed into a compound without the protective group using standard procedures or a reagent such as formic acid.

d1) Compounds of the general formula (IV) which are defined as above can be formed by reacting a compound of formula (VI) using standard conditions or with a chlorinating reagent such as thionyl chloride or POCl₃. Advantageously dimethylformamide may be used. The reaction may be performed in an inert solvent. Advantageously the inert solvent is toluene.

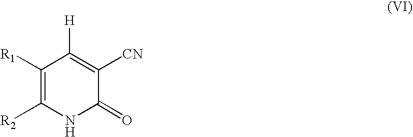

d2) Compounds of the general formula (IV) in which R₁ is as defined above, R₂ is methyl or ethyl and L is a chloro group can be formed by reacting a compound of formula (IX),

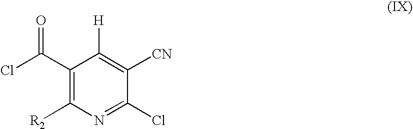

in which R₂ is methyl or ethyl, with R₆OH wherein R₆ is as defined in formula (I).
The reaction is generally carried out in an inert solvent, such as THF.
Optionally the reaction is carried out in the precence of a suitable organic base, such as TEA or DIPEA.

The preparation of compounds of the general formula (IX) which is defined as above comprises the steps (e1-e4) below;

e1) Reacting a compound of the formula (X), in which R₂ is methyl or ethyl and R′ is a carboxylic acid protective group such as an alkyl or a benzyl group, with dimethoxy-N,N-dimethylmethaneamine to form a

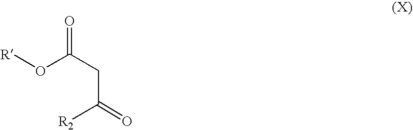

compound of formula (XI).

e2) This compound (XI) can then be reacted further with a compound of the

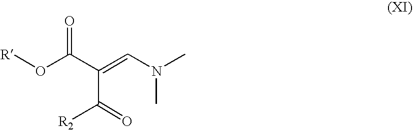

general formula NC—CH₂C(O)NH₂ to give a compound of the general formula (XII). The reaction is generally performed in an inert solvent such as ethanol, optionally in the presence of a strong base such as sodium ethoxide.

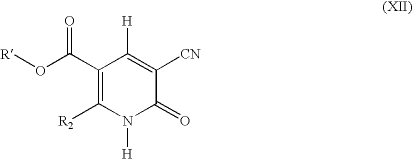

e3) A compound of the general formula (XII) can then be transformed to a compound of the general formula (XIII). The reaction is generally performed in a protic solvent such as water together with a co-solvent such as THF or methanol. The reaction can be performed using standard reagents or in the presence of LiOH, NaOH or KOH.

e4) Compounds of general formula (IX) is then formed by reacting a compound of formula (XIII) in which R₂ is methyl or ethyl,

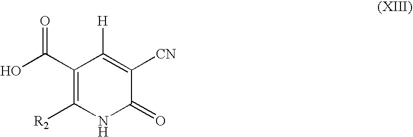

with a chlorinating agent such as thionyl chloride, POCl₃ or oxalyl chloride.
Usually the reaction is carried out at room temperature or at elevated temperatures in an inert solvent such as CH₂Cl₂.
Occasionally the reaction is performed in the presence of DMF to catalyze the reaction.

(f) The preparation of compounds of the general formula (VII) which is defined as above comprises the steps (f1-f3) or (f4-f6) below;

(f1) Reacting a compound of formula (XIV)

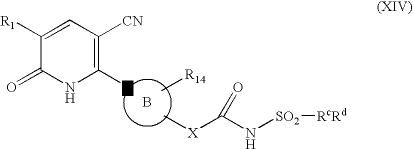

wherein R₁, B, X, R₁₄, R^{c} and R^{d} are defined as with a halogenating reagent such as POCl₃ or with trifluoromethanesulfonic anhydride (triflic anhydride).

The reaction is usually carried out in an inert solvent such as CH₂Cl₂.
Optionally the reaction is performed in the presence of an organic base such as TEA and DIPEA.

f2) Compounds of general formula (XIV) above may be prepared by reacting a compound of the general formula (XV)

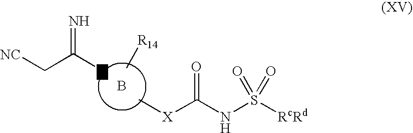

where B, R₁₄, X, R^{c} and R^{d} are as defined in formula (I) above with a compound of formula (XVI)

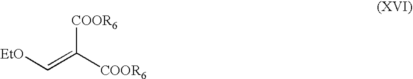

The reaction is generally carried out in an inert organic solvent such as EtOH or DMSO.

The reaction is carried out at ambient temperature or at elevated temperatures using standard equipment or a single node microwave oven.

f3) Compounds of the general formula (XV) defined above can be prepared by reacting a compound of the general formula (V) as defined above with a compound of formula (XVII)

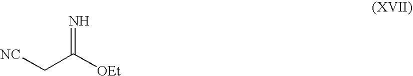

using essentially the same procedure as described in [Macconi, A et. Al., J. Heterocyclic chemistry, 26, p. 1859 (1989)].

f4) Compounds of general formula (VII) above may also be prepared by reacting a compound of the general formula (XVIII),

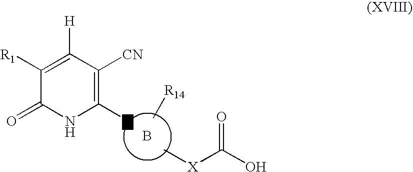

wherein R₁, R₁₄, X and B are defined as in formula (I), with a compound of formula (III) as defined above.
The reaction is generally carried out in an inert organic solvent such as dichloromethane at ambient temperature. The reaction may be carried out using standard conditions or in the presence of TBTU, EDCI, PyBrop or the combination of EDCI and HOBT. Optionally, the reaction may be carried out in the presence of an organic base such as triethylamine or DIPEA.

f5) Compounds of general formula (XVIII) above may be prepared by reacting a compound of the general formula (XIX)

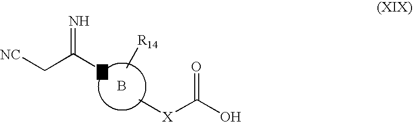

where B, R₁₄ and X, defined as in formula (I) above with a compound of formula (XVI) as defined above.

f6) Compounds of the general formula (XIX) defined above can be prepared by reacting a compound of the general formula (VIII) as defined above with a compound of formula (XVI)

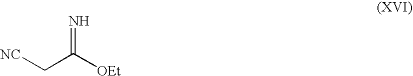

using essentially the same procedure as described in [Macconi, A et. Al., J. Heterocyclic chemistry, 26, p. 1859 (1989)].

g) The preparation of compounds of the general formula (XX), in which R₁₄ is defined as for formula (I) with the exception that R₁₄ is connected to the same atom as X, and X is defined as a single bond, comprises the below step;

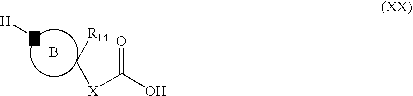

h) Reacting the corresponding (XXI) with R₁₄-L, wherein L is a suitable leaving group, such as chloro, bromo, iodo,

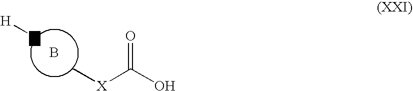

triflate (OTf) or tosyl (OTs) to form compounds of the general formula (XXI), using standard conditions or in the presence of with BuLi and diisopropylamine mixture. The preparation of compounds of the formula (III) comprises the below processes. (i1-i3)

i1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column.) followed by hydrolysis using a base like NaOMe in an inert solvent like DMSO at room temperature. Followed by treatment by NH₂OSO₃H and NaOAc to give a compound of formula (III).

i2) A compound of the formula LSO₂R^{c}R^{d} wherein L is a suitable leaving group, such as chloro, bromo, iodo could be reacted with ammonium hydroxide in an inert solvent such as DCM or THF to give a compound of formula (III).

i3) A compound of the formula LR^{c}R^{d} wherein L is a suitable leaving group, such as chloro, bromo, iodo could be transformed to the corresponding compound (III) using a sequence of reactions first NaSO₃, followed by a using a reagent such as PCl₅, POCl₃ or SOCl₂, followed by ammonium hydroxide to give a compound of formula (III).

Persons skilled in the art will appreciate that an acid can be transformed to the corresponding activated ester such as an acid chloride, followed by reaction with a alcohol, R₆OH to give esters, R₆OC(O).

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscince (1999).

Protected derivatives of the invention may be converted chemically to compounds of the invention using standard deprotection techniques (e.g. under alkaline or acidic conditions). The skilled person will also appreciate that certain compounds of Formula (II)-(XXI) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diasteromeric derivatives by conventionals means (e.g. HPLC, chromatography over silica or crystallization). Stereocenters may also be introduced by asymmetric synthesis, (e.g metalloorganic reactions using chiral ligands). All stereoisomers are included within the scope of the invention.

It will also be understood that some of the compounds described in the processes above may exhibit the phenomenon of tautomerism and the processes described above includes any tautomeric form.

All novel intermediates form a further aspect of the invention.

### Pharmacological Data

Functional inhibition of—the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.
Most of the compounds of the invention have an activity, when tested in the functional inhibition of 2-Me-S-ADP induced P2Y₁₂ signalling assay described, at a concentration of around 2 μM or below.

For example the compounds described in Examples 2 and 5 gave the following test result in the functional inhibition of 2-Me-S-ADP induced P2Y₁₂ signalling assay described.

| | IC₅₀(μM) |
|---|---|
| Example 2 | 0.12 |
| Example 5 | 0.07 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

Thus, according to another further aspect of the invention there is provided a compound of formula (I), or a pharmaceutically acceptable salt thereof, for use as a medicament.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

In yet another aspect of the invention there is provided a compound of formula (I), or a pharmaceutically acceptable salt thereof, for use as an inhibitor of the P2Y₁₂ receptor.

In still another aspect of the invention there is provided a compound of formula (I), or a pharmaceutically acceptable salt thereof, for use in the treatment of platelet aggregation disorder.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-MS) or LC-MS system consisting of a Waters ZQ using a LC-Agilent 1100 LC system. ¹H NMR measurements were performed on a Varian Mercury VXR 300 and VX 400 spectrometer, operating at a 1H frequency of 300 and 400 MHz and Varian UNITY plus 400, 500 and 600 spectrometers, operating at 1H frequencies of 400, 500 and 600 MHz respectively. Chemical shifts are given in ppm with the solvent as internal standard. Protones on heteroatoms such as NH and OH protons are only reported when detected in NMR and can therefore be missing. HPLC separations were performed on a Waters YMC-ODS AQS-3 120 Angstrom 3×500 mm or on a Waters Delta Prep Systems using Kromasil C8, 10 μm columns.

Purification Method A: The purification system and LC-MS system used in purification Method A, referred to in some of the examples below, was Waters Fraction Lynx I Purification System Column: Sunfire Prep C₁₈, 5 μm OBD, 19×150 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.

Reactions performed in a microwave reactor were performed in a Personal Chemistry Smith Creator, Smith synthesizer or an Emrys Optimizer.

IUPAC names were generated with ACDLabs Name, Release 9:00, Product version 9.04.

The GTPγS values (IC50 in μM) mentioned in the examples below were measured by the method described below:

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

### LIST OF USED ABBREVIATIONS

| Abbreviation | Explanation |
|---|---|
| aq | Aqueous |
| br | Broad |
| Brine | A saturated solution of sodium chloride |
| | in water |
| BSA | Bovine Serum Albumine |
| d | Doublet |
| DCM | Dichloromethane |
| DIPEA | N,N-Diisopropylethylamine |
| DMA | N,N-Dimethylacetamide |
| DMAP | N,N-Dimethylpyridin-4-amine |
| DMF | N,N-dimethylformamide |
| DMSO | Dimethylsulphoxide |
| EtOAc | Ethyl acetate |
| EtOH | Ethanol |
| HEPES | [4-(2-hydroxyethyl)-1- |
| | piperazineethanesulfonic acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| J | Coupling constant |
| LC | Liquid chromatography |
| m | Multiplet |
| MHz | Megahertz |
| mL | Millilitre |
| mM | Millimolar |
| MS | Mass spectra |
| NMR | Nuclear magnetic resonance |
| OAc | acetate |
| PyBrop | Bromo(tripyrrolidin-1-yl)phosphonium |
| | hexafluorophosphate |
| q | Quartet |
| r.t | Room temperature |
| s | Singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | Trifluormethanesulfonyl |
| TFA | Trifluoroacetic acid |
| THF | Tetrahydrofurane |
| TMEDA | N,N,N′,N′-tetramethylethylendiamine |
| Ts | Para toluenesulfonyl |

### Sulfone Amides

### Synthesis of Sulfone Amides

The synthesis of the sulfonamides used in the examples below was made with one of the three methods described below:

i) By reacting the corresponding sulfonyl chloride with ammonia in THF or MeOH or by treatment with ammonium hydroxide in methylene chloride. The sulfonamides obtained were used without further purification.

ii) By essentially following the procedure described by Seto, T. et. al. in J. Organic Chemistry, Vol 68, No 10 (2003), pp. 4123-4125. or

iii) By essentially following the procedure described by Wang, Z et. al. in Tetrahedron Letters, Vol 43 (2002), pp 8479-8483.

### Example 1

### Ethyl 6-(3-{2-[(benzylsulfonyl)amino]-2-oxoethyl}pyrrolidin-1-yl)-5-cyano-2-methylnicotinate

### (a) Ethyl 2-((dimethylamino)methylene)-3-oxobutanoate

1,1-dimethoxy-N,N-dimethylmethanamine (500 g, 4195 mmol) was added to ethyl 3-oxobutanoate (461.6 g, 3547 mmol) under an atmosphere of nitrogen at r.t during 13 minutes (weak exotherm). The orange red solution was stirred for 22 hours and concentrated in vaccuo. The residue was co-evaporated with toluene (3 times 200 ml each) and used without no need for further purification in the next step.

MS m/Z: 186 (M+1).

### (b) Ethyl 5-cyano-2-methyl-6-oxo-1,6-dihydropyridine-3-carboxylate

Sodium ethoxide (1240.7 g of a 21 wt % solution in EtOH, 3829 mmol) was added to a stirred suspension of 2-cyanoacetamide (298 g, 3544 mmol) in EtOH (3000 mL) during 8 minutes under an atmosphere of nitrogen at r.t. The crude condensation product from step (a) above dissolved in 950 ml EtOH was added slowly (slightly exothermic reaction) and after about one third had been added further EtOH (1000 mL) was added to allow efficient stirring (suspension) followed by the addition of the rest of the condensation product (total addition time 30 min). After stirring over night at r.t. HOAc (526 g, 8759 mmol) was added to the reaction and the mixture was concentrated in vaccuo leaving a thick orange slurry (volume about 3000 mL), 1 M HCl (4628 mL, 4628 mmol) was added during 10 min followed by water (500 mL). The stirring was stopped and the precipitate was filtered off and washed with water (200 mL). NMR showed the presence of about 5-10% of the corresponding acid and the solid was washed by stirring with further water (1500 mL+3×1000 mL), a solution of saturated NaHCO₃ (400 mL) in water (600 mL) and finally water (1000 mL). Filtration of the solid and drying in vaccuo at 80° C. gave pure ethyl 5-cyano-2-methyl-6-oxo-1,6-dihydropyridine-3-carboxylate. Yield: 493 g (67%).

¹H NMR (400 MHz, DMSO-d₆): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylnicotinate

Toluene (4000 mL) and thionylchloride (507 g, 4262 mmol) were added to ethyl 5-cyano-2-methyl-6-oxo-1,6-dihydropyridine-3-carboxylate (293 g, 1421 mmol) under an atmosphere of nitrogen and the mixture was heated to 50° C. (oil bath temperature) and DMF (100 g, 1368 mmol) was added during 2 minutes. The temperature was raised to 80° C. (oil bath temperature) and the stirring was continued for 2 hours. The mixture was concentrated in vaccuo (about 3500 ml was evaporated off) leaving a red oil. EtOH (1000 mL, 99%) was added and then evaporated off. Dichloromethane (4000 mL) was added followed by 4% NaHCO₃ solution (4000 mL) and the mixture was stirred for 15 minutes. The organic phase was separated and evaporated to give ethyl 6-chloro-5-cyano-2-methylnicotinate as a dark red crude solid which was used without further purification. Yield: 301 g (75%).

¹H NMR (400 MHz, CDCl₃): δ 1.42 (3H, t, J=7.1 Hz), 2.91 (3H, s), 4.40 (2H, q, J=7.1 Hz), 8.49 (1H, s).

### (d) {1-[3-cyano-5-(ethoxycarbonyl)-6-methylpyridin-2-yl]pyrrolidin-3-yl}acetic Acid

A mixture of ethyl 6-chloro-5-cyano-2-methylnicotinate (270 mg, 1.2 mmol), pyrrolidin-3-ylacetic acid (155 mg, 1.2 mmol) and triethylamine (243 mg, 2.4 mmol) in 95% ethanol (5 mL) was subjected to single node microwave heating, at 120 C for 10 minutes followed by an additional 15 minutes at 150° C. Evaporation of the solvent gave a crude product as a light brown solid (633 mg). Purification with HPLC (19×250 mm, Kromasil C₈, 10 μm column at 30 mL/minute using a stepwise gradient from 5% CH₃CN to 50% during 25 min) gave 296 mg of a pale yellow oil that solidified upon standing. NMR analysis confirmed the desired product but also the presence of about 25% starting material. The product mixture was therefore charged into a Microwave vessel together with pyrrolidin-3-ylacetic acid (77.5 mg, 0.6 mmol) and triethylamine (121.5 mg, 1.2 mmol) and ethanol and heated for 15 minutes at 150° C. Purification with HPLC (19×250 mm, Kromasil C₈, 10 μm column at 30 mL/minute using a stepwise gradient from 5% CH₃CN to 50% during 25 minutes) gave the product as a light yellow solid (80% pure). This material was used without further purification in the next step. Yield: 272 mg (57%).

### (e) Ethyl 6-(3-{2-[(benzylsulfonyl)amino]-2-oxoethyl}pyrrolidin-1-yl)-5-cyano-2-methylnicotinate

DIPEA (129 mg, 1 mmol) and TBTU (77 mg, 0.24 mmol) was added to a solution of {1-[3-cyano-5-(ethoxycarbonyl)-6-methylpyridin-2-yl]pyrrolidin-3-yl}acetic acid (69.8 mg, 0.22 mmol) in DCM (2 mL) at room temperature. The reaction was stirred for 10 minutes followed by the addition of 1-phenylmethanesulfonamide (34.2 mg, 0.20 mmol) and the reaction mixture was stirred over night. Addition of 0.1 M KHSO₄ (2 mL) and collection of the organic phase using a phase separator gave a crude product which was purified by preparative HPLC using Waters Fraction Lynx Purification System with Kromasil C₈, 5 μm 20×100 mm columns. The mobile phase used was a gradient of acetonitrile and 0.1 mM ammonium acetate buffer. The flow was 30 mL/minute. MS triggered fraction collection was used. Mass spectra were recorded on either a Micromass ZQ single quadrupole or a Micromass Quattro micro, both equipped with a pneumatically assisted electro spray interface. Yield: 8.8 mg (9.4%).

¹H NMR (600 MHz, DMSO-d₆) δ 1.27 (3H, t, J=7.0 Hz), 1.57-1.66 (1H, m), 2.07-2.15 (1H, m), 2.38-2.42 (1H, m), 2.45-2.48 (1H, m), 2.60 (3H, s), 3.64-3.73 (1H, m), 3.80-3.89 (1H, m), 3.91-4.03 (1H, m), 4.20 (2H, q, J=7.1 Hz), 4.67 (2H, s), 7.27-7.31 (2H, m), 7.33-7.40 (3H, m), 8.26 (1H, s), 11.55-11.60 (1H, m)

MS m/Z: 471 (M+1), 469 (M−1)

GTPγS(IC₅₀ μM): 0.143

### Example 2

### Ethyl 5-cyano-2-methyl-6-[3-(2-oxo-2-{[(2-phenylethyl)sulfonyl]amino}ethyl)pyrrolidin-1-yl]nicotinate

Prepared according to the procedure described in Example 1 from {1-[3-cyano-5-(ethoxycarbonyl)-6-methylpyridin-2-yl]pyrrolidin-3-yl}acetic acid and 2-phenylethanesulfonamide to give ethyl 5-cyano-2-methyl-6-[3-(2-oxo-2-{[(2-phenylethyl)sulfonyl]amino}ethyl)pyrrolidin-1-yl]nicotinate. Yield: 2.5 mg (2.6%).

MS m/Z: 485 (M+1), 483 (M−1).

GTPγS(IC₅₀ μM): 0.12

### Example 3

### 2,2,2-Trifluoroethyl 5-cyano-6-(4-{1-[(4-fluorobenzyl)sulfonyl]carbamoyl}piperidin-1-yl)-2-methylnicotinate

### (a) 5-Cyano-2-methyl-6-oxo-1,6-dihydropyridine-3-carboxylic Acid

KOH (1.43 g, 25.5 mmol) dissolved in EtOH (25 mL, 95%) was added to ethyl 5-cyano-2-methyl-6-oxo-1,6-dihydropyridine-3-carboxylate (1.69 g, 8.2 mmol) in EtOH (30 mL) to give a thick slurry which was heated to reflux (90 degrees in the oil bath) for 12 hours. The mixture was concentrated and 2 M HCl was added. The precipitate formed was filtered, washed with water and dried to give 5-cyano-2-methyl-6-oxo-1,6-dihydropyridine-3-carboxylic acid as a white solid. Yield: 1.425 g (98%).

1H NMR (500 MHz, DMSO-d₆): keto-form: 2.61 (3H, s), 8.40 (1H, s), 12.91 (1H, br s). ˜86% and enol-form: 2.36 (3H, s), 8.09 (1H, s), 10.50 (1H, br s). ˜14%

MS m/Z: 179 (M+1), 177 (M−1).

### (b) 6-Chloro-5-cyano-2-methylnicotinoyl Chloride

Oxalylchloride (3.38 mL, 40 mmol) was added dropwise to a cold (ice/water bath) suspension of 5-cyano-2-methyl-6-oxo-1,6-dihydropyridine-3-carboxylic acid (0.710 g, 3.99 mmol) in dry DCM (25 mL) followed by dry DMF (0.1 mL). The reaction was stirred for 20 minutes at 0 degrees and then at room temperature for 30 minutes followed by reflux for 16 hours. The mixture was evaporated and the remaining black residue was co-evaporated with dry DCM (two times). The crude product was used in the next step without further purification.

### (c) 2,2,2-Trifluoroethyl 6-chloro-5-cyano-2-methylnicotinate

DIPEA (14.22 g, 110 mmol) was added dropwise to a stirred solution of 6-chloro-5-cyano-2-methylnicotinoyl chloride (4.73 g, 22 mmol) and 2,2,2,-trifluoroethanol (22.0 g, 220 mmol) in dry THF (100 mL) under an atmosphere of nitrogen. The stirring was continued for 18 hours at r.t. and the solvent removed in vaccuo. The residue was dissolved in DCM (120 mL) and the organic phase was washed with NaHCO₃(sat,aq) (2×120 mL) and water (120 mL). The water phase was extracted with DCM (2×50 mL) and the combined organic phase was dried (MgSO₄), filtered and evaporated to give 2,2,2-trifluoroethyl 6-chloro-5-cyano-2-methylnicotinate which was used without further purification in the next step.

### (d) 1-{3-Cyano-6-methyl-5-[(2,2,2-trifluoroethoxy)carbonyl]pyridin-2-yl}piperidine-4-carboxylic Acid

A solution of 2,2,2-trifluoroethyl 6-chloro-5-cyano-2-methylnicotinate (3.07 g, 110 mmol), piperidine-4-carboxylic acid (1.42 g, 110 mmol) and DIPEA (1.96 mL, 113 mmol) in DMF (50 mL) was heated at 90° C. for 1 hour. The solvent was removed in vaccuo and the residue was dissolved in DCM (150 mL) and washed with NH₄Cl(aq,sat) (150 mL). The water phase was extracted with DCM (3×50 mL) and the combined organic phase was washed with water (50 mL), dried, filtered and evaporated to give a crude product which was purified by preparative HPLC (Kromasil C₈, 110 μm using a gradient of CH₃CN/0.1 M NH₄OAc(aq)) to give the pure product. Yield: 70 mg (1.7%)

¹H NMR (400 MHz, CDCl₃): δ 1.74-1.86 (2H, m), 1.97-2.06 (2H, m), 2.64 (3H, s), 2.60-2.68 (1H, m), 3.21-3.33 (2H, m), 4.51-4.60 (4H, m), 8.28 (1H, s).

### (e) 2,2,2-Trifluoroethyl 5-cyano-6-(4-{[(4-fluorobenzyl)sulfonyl]carbamoyl}piperidin-1-yl)-2-methylnicotinate

1-(4-fluorophenyl)methanesulfonamide (33.6 mg, 0.178 mmol) was added after 30 minutes to a mixture of 1-{3-cyano-6-methyl-5-[(2,2,2-trifluoroethoxy)carbonyl]pyridin-2-yl}piperidine-4-carboxylic acid (66 mg, 0.178 mmol), TBTU (85.6 mg, 0.267 mmol) and DIPEA (115 mg, 0.889 mmol) in DCM at r.t. and the reaction was stirred for 1 hour. An additional amount of TBTU (28.5 mg, 0.089 mmol) and 1-(4-fluorophenyl)methanesulfonamide (20 mg, 0.053 mmol) was added and the stirring was continued for 1 hour at r.t. followed by addition of DCM (20 mL), NH₄Cl(aq,sat) (20 mL) and 3 M HCl to lower the pH of the solution. Drying of the organic phase(MgSO₄), filtration and evaporation gave a crude product which was purified by preparative HPLC (Kromasil C₈ 10 μm, using a gradient of CH₃CN/0.2% HOAc in water) to give the pure product after freeze drying as a powder. Yield: 56 mg (58%).

¹H NMR (500 MHz, DMSO-d₆): δ 1.57-1.70 (2H, m), 1.81-1.90 (2H, m), 2.5-2.65 (1H, m), 2.64 (3H, s), 3.12-3.22 (2H, m), 4.54-4.62 (2H, m), 4.69 (2H, s), 4.87-4.97 (2H, m), 7.20-7.27 (2H, m), 7.29-7.36 (2H, m), 8.33 (1H, s), 11.61 (1H, br.s, NH).

GTPγS(IC₅₀ μM): 0.024

### Example 4

### Ethyl 5-cyano-6-(3-{[(2-fluoro-5-methylbenzyl)sulfonyl]carbamoyl}azetidin-1-yl)-2-methylnicotinate

### (a) 1-[3-Cyano-5-(ethoxycarbonyl)-6-methylpyridine-2-yl]azetidine-3-carboxylic Acid

Ethyl 6-chloro-5-cyano-2-methylnicotinate (50.98 g, 227 mmol), azetidine-3-carboxylic acid (24.09 g, 238 mmol) and DIPEA (118.9 mL, 681 mmol) were suspended in EtOH (250 mL) and heated at reflux for 1 h. The reaction mixture was cooled to r.t and added drop-wise to KHSO₄ (154.5 g, 1135 mmol) in water (3000 mL). The solids were collected by filtration and dried under vacuum to afford 1-[3-Cyano-5-(ethoxycarbonyl)-6-methylpyridine-2-yl]azetidine-3-carboxylic acid as a solid, which was used without further purification. Yield: 65.33 g (100%).

¹H NMR (400 MHz, CDCl₃): δ 1.37 (3H, t, J=7.1 Hz), 2.72 (3H, s), 3.59-3.68 (1H, m), 4.31 (2H, q, J=7.1 Hz), 4.55-4.68 (4H, m), 8.28 (1H, s).

MS m/Z: 290 (M+1).

### (b) Ethyl 5-cyano-6-(3-{[(2-fluoro-5-methylbenzyl)sulfonyl]carbamoyl}azetidin-1-yl)-2-methylnicotinate

1-(2-fluoro-5-methylphenyl)methanesulfonamide (20.3 mg, 0.1 mmol) dissolved in 1/1 DCM/DMF (1 mL) was added after 20 minutes to a solution of 1-[3-cyano-5-(ethoxycarbonyl)-6-methylpyridine-2-yl]azetidine-3-carboxylic acid (29.9 mg, 0.1 mmol), TBTU (48.2 mg, 0.15 mmol) and DIPEA (65 mg, 0.5 mmol) in 1/1 DCM/DMF (3 mL) and the mixture was stirred at r.t. over night. LC-MS showed that starting material was left and DIPEA (65 mg, 0.5 mmol) and DMAP (2.44 mg, 0.2 mmol) was added and the mixture stirred for another 2 days. Still some remaining starting material and therefore PyBrop (46.6 mg, 0.1 mmol) was added and the stirring was continued over night. Removal of the solvent followed by purification by preparative HPLC (Kromasil C₈ 10 μm, using a gradient of CH₃CN/0.1 M NH₄OAc in water) to give the pure product after freeze drying. Yield: 11.5 mg (24%).

¹H NMR (500 MHz, DMSO-d₆): δ 1.29 (3H, t, J=7.1 Hz), 2.27 (3H, s), 2.63 (3H, s), 3.52-3.62 (1H, m), 4.23 (2H, q, J=7.1 Hz), 4.30-4.37 (2H, m), 4.72 (2H, s), 7.07-7.15 (1H, m), 7.18-7.26 (2H, m), 8.30 (1H, s), 11.94 (1H, br. S, NH).

MS m/Z: 476 (M+1).

GTPγS(IC₅₀ μM): 0.018

### Example 5

### Ethyl 6-{4-[(benzylsulfonyl)carbamoyl]-3-methylpiperidin-1-yl}-5-cyano-2-methylnicotinate

### (a) 1-[3-Cyano-5-(ethoxycarbonyl)-6-methylpyridin-2-yl]-3-methylpiperidine-4-carboxylic Acid

DIPEA (371 mg, 2.87 mmol) was added to a solution of ethyl 6-chloro-5-cyano-2-methylnicotinate (260 mg, 1.157 mmol) and 3-methylpiperidine-4-carboxylic acid hydrochloride (200 mg, 1.113 mmol) and the mixture was heated to 120° C. for 5 minutes using microwave single node heating. NH₄Cl(aq,sat) was added and the mixture was extracted with DCM (3 times). The combined organic layer was run through a phase separator and evaporated to give the crude product was used in the next step without further purification.

MS m/Z: 332 (M+1), 330 (M−1).

### (b) Ethyl 6-{4-[(benzylsulfonyl)carbamoyl]-3-methylpiperidin-1-yl}-5-cyano-2-methylnicotinate

1-(phenyl)methanesulfonamide (190 mg, 1.11 mmol) was added after 2 hours to a stirred solution of 1-[3-Cyano-5-(ethoxycarbonyl)-6-methylpyridin-2-yl]-3-methylpiperidine-4-carboxylic acid (367.8 mg, 1.11 mmol), TBTU (408 mg, 1.27 mmol) and DIPEA (297 mg, 2.30 mmol) in DCM (5 mL) and the stirring was continued for 18 hours at r.t. NaHCO₃(aq) was added and the mixture was extracted with DCM (3 times). The combined organic layer was run through a phase separator, evaporated and the crude product was purified by preparative HPLC (Kromasil C₈ 10 μm, 21.5×250 mm flow 25 mL/minute, using a gradient of CH₃CN/0.1 M NH₄OAc) to give the product after freeze drying. Yield: 334 mg (62%).

¹H NMR (500 MHz, DMSO-d₆): δ 0.84 (3H, d, J=7.1 Hz), 1.30 (3H, t, J=7.1 Hz), 1.67-1.73 (1H, m), 1.84-1.92 (1H, m), 2.24-2.30 (1H, m), 2.64 (3H, s), 2.67-2.72 (1H, m), 3.39-3.44 (1H, m), 3.44-3.49 (1H, m), 4.25 (2H, q, J=7.1), 4.28-4.32 (1H, m), 4.33-4.38 (1H, m), 4.70 (2H, s), 7.31-7.34 (2H, m), 7.37-7.43 (3H, m), 8.32 (1H, s), 11.63 (1H, s).

MS m/Z: 485 (M+1), 483 (M−1).

GTPγS(IC₅₀ μM): 0.067

### Example 6

### Ethyl 6-{4-[(benzylsulfonyl)carbamoyl]piperidin-1-yl}-5-cyano-2-(methylamino)nicotinate

### (a) tert-Butyl 4-{[(benzylsulfonyl)amino]carbonyl}piperidine-1-carboxylate

TEA (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vaccuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBt that was filtered off. Most of the solvent was removed in vaccuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25 oC to give tert-butyl 4-[(benzylsulfonyl)carbamoyl]piperidine-1-carboxylate as a white solid. Yield 584 g (78%).

¹H NMR (400 MHz, CDCl₃): δ 1.46 (9H, s), 1.54-1.61 (2H, m), 1.70-1.74 (2H, m), 2.19-2.27 (1H, m), 2.68-2.75 (2H, m), 4.07-4.12 (2H, m), 4.66 (2H, s), 7.32-7.41 (5H, m), 7.54 (1H, br s).

### (b) tert-Butyl 4-[allyl(benzylsulfonyl)carbamoyl]piperidine-1-carboxylate

A mixture of tert-butyl 4-[(benzylsulfonyl)carbamoyl]piperidine-1-carboxylate (11.47 g, 30 mmol), 3-bromoprop-1-ene (10.89 g, 90 mmol) and DIPEA (7.76 g, 60 mmol) in DMF (30 mL) was stirred at r.t. for 21 hours. Water (75 mL) was added and the aqueous phase was extracted with heptane/DCM 4/1 (3×75 mL). The combined organic phase was dried (MgSO₄), filtered and evaporated to give the product which was used without further purification.

### (c) N-allyl-N-(benzylsulfonyl)piperidine-4-carboxamide trifluoroacetate

TFA/DCM 2/1 (30 mL) was added to a stirred solution of tert-butyl 4-[allyl(benzylsulfonyl)carbamoyl]piperidine-1-carboxylate (12.676 g, 30 mmol) in DCM (10 mL) at 0° C. (ice/water bath) and the stirring was continued for 5 minutes followed by 4 hours at r.t. The solvent was evaporated and the mixture was co-evaporated with DCM twice to give the product as a TFA salt which was used in the next step without further purification.

### (d) N-allyl-N-(benzylsulfonyl)-1-(2-cyanoethanimidoyl)piperidine-4-carboxamide

N-allyl-N-(benzylsulfonyl)piperidine-4-carboxamide trifluoroacetate (30 mmol) was added to a cold (ice/water bath temperature) solution of ethyl 2-cyanoethanimidoate (See McElvain, S. M.; Schroeder, J. P.; J. Am. Chem. Soc. 71, p. 40 (1949)) (15.14 g, 101.25 mmol, 75% pure) and DIPEA (23.26 g, 180 mmol) in EtOH (200 mL) and the mixture was stirred for 10 minutes followed by 16 hours at r.t. LC-MS showed complete conversion of the starting material. This solution was used in the next step as such.

### (e) Ethyl 6-{4-[allyl(benzylsulfonyl)carbamoyl]piperidin-1-yl}-5-cyano-2-oxo-1,2-dihydropyridine-3-carboxylate

Diethyl (ethoxymethylene)malonate (8.43 g, 39 mmol) was added to the solution from step (d) above and the reaction mixture was stirred for 18 hours at r.t. Evaporation of the solvent gave 32 g of a crude product. 8 g (¼) of this was taken out and purified by preparative HPLC (Kromasil C₈ 10 μm, Eluent: A: CH₃CN; B: 0.2% HOAc in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give two fractions containing the product. Fraction 1: 308 mg (8% chemical yield, 100% purity according to LC-MS and Fraction 2: 853 mg (76% pure according to LC-MS).

¹H-NMR (400 MHz, CDCl₃): δ 1.40 (3H, t, J=7.2 Hz), 1.57-1.80 (4H, m), 2.60-2.70 (1H, m), 2.92-3.03 (2H, m), 4.11-4.16 (2H, m), 4.39/2H, q, J=7.2 Hz), 4.61 (2H, s), 4.64-4.72 (2H, m), 5.19-5.30 (2H, m), 6.62-5.75 (1H, m), 7.31-7.45 (5H, m), 8.24 (1H, s), 11.90 (1H, br. S, NH).

### (f) Ethyl 6-{4-[allyl(benzylsulfonyl)carbamoyl]piperidin-1-yl}-5-cyano-2-{[(trifluoromethyl)sulfonyl]oxy}nicotinate

Trifluoromethanesulfonic anhydride (186 mg, 0.66 mmol) was added dropwise to a cold (ice/water bath temperature) solution of ethyl 6-{4-[allyl(benzylsulfonyl)carbamoyl]piperidin-1-yl}-5-cyano-2-oxo-1,2-dihydropyridine-3-carboxylate (308 mg, 0.6 mmol) and TEA (273 mg, 2.7 mmol) in DCM (7 mL). The reaction was stirred at 0° C. for 1 hour and NaHCO₃ (aq,sat) was added. The aqueous phase was extracted with DCM (2×10 mL). The combined organic phase was dried (Na₂SO₄), filtered and evaporated to give the product which was used without further purification.

### (g) Ethyl 6-{4-[allyl(benzylsulfonyl)carbamoyl]piperidin-1-yl}-5-cyano-2-(methylamino)nicotinate

To ethyl 6-{4-[allyl(benzylsulfonyl)carbamoyl]piperidin-1-yl}-5-cyano-2-{[(trifluoromethyl)sulfonyl]oxy}nicotinate (100 mg, 0.135 mmol) in a Smith process vial was added A 2M solution of methylamine in THF (2 mL) and the mixture was stirred at r.t. for 16 hours followed by 100° C. for 10 minutes using microwave single node heating. LC-MS showed that there was some starting material left and therefore DIPEA (152 mg, 1.176 mmol) was added and the mixture was heated to 110° C. for 30 minutes using microwave single node heating followed by 120° C. for 30 minutes. The crude product obtained after evaporation of the solvent and excess reactants was used as such in the next step.

### (h) Ethyl 6-{4-[(benzylsulfonyl)carbamoyl]piperidin-1-yl}-5-cyano-2-(methylamino)nicotinate

Sodium 4-methylbenzenesulfinate (40 mg, 0.224 mmol) and Pd(PPh₃)₄ (7 mg, 0.006 mmol) was added to a solution of ethyl 6-{4-[allyl(benzylsulfonyl)carbamoyl]piperidin-1-yl}-5-cyano-2-(methylamino)nicotinate (74 mg, 0.083 mmol) in DCM (2 mL) and the mixture was stirred at r.t. for 14 hours. An additional amount of Pd(PPh₃)₄ (10 mg, 0.009 mmol) was added and the stirring was continued for an additional 2 hours at r.t. Evaporation of the solvent gave a crude product which was purified by preparative HPLC (Kromasil C₈, 110 μm using a gradient of CH₃CN/0.1 M HOAc in water) to give the pure product after freeze drying. Yield: 18 mg (45%).

¹H NMR (400 MHz, CDCl₃): δ 1.36 (3H, t, J=7.1 Hz), 1.73-1.90 (4H, m), 2.36-2.47 (1H, m), 3.01 (3H, d, J=4.8 Hz), 3.04-3.13 (m, 2H), 4.27 (2H, q, J=7.1 Hz), 4.59-4.65 (2H, m), 4.66 (2H, s), 7.32-7.36 (2H, m), 7.37-7.42 (3H, m), 8.20 (1H, s), 8.33 (1H, br q, J=4.8 Hz).

MS m/Z: 486 (M+1)

GTPγS(IC₅₀ μM): 0.044

### Example 7

### Ethyl 5-cyano-6-(4-{[(2-fluoro-5-methylbenzyl)sulfonyl]carbamoyl}piperidin-1-yl)-2-methylnicotinate

### (a) 1-[3-cyano-5-(ethoxycarbonyl)-6-methylpyridin-2-yl]piperidine-4-carboxylic Acid

Ethyl 6-chloro-5-cyano-2-methylnicotinate (3.00 g, 13.35 mmol), piperidine-4-carboxylic acid (1.897 g, 14.69 mmol), and TEA (2.703 g, 26.71 mmol) were mixed and the mixture was refluxed for 10 minutes. LC/MS showed full conversion. The reaction mixture was evaporated, water/EtOAc 1:1 (100 mL) was added and the water phase was acidified to pH3. The EtOAc phase was separated and the water phase was extracted with an additional EtOAc (40 mL). The combined organic phases were dried (Na₂SO₄), filtered and evaporated to give 3.8 g of a crude material.

Purification with preparative HPLC (Kromasil C₈, 10 μm at pH=7 (0.1 M NH₄OAc/CH₃CN) with subsequent switch to pH=3) gave the pure product. Yield: 1.9 g (45

¹H NMR (400 MHz, CDCl₃): δ 1.38 (t, J=7.1 Hz, 3H), 1.94-1.82 (m, 2H), 2.13-2.05 (m, 2H), 2.75-2.66 (m, 5H), 3.37-3.27 (m, 2H), 4.33 (q, J=7.1 Hz, 2H), 4.63-4.55 (m, 2H), 8.36 (s, 1H)

MS m/Z: 318 (M+1).

### (b) Ethyl 5-cyano-6-(4-{[(2-fluoro-5-methylbenzyl)sulfonyl]carbamoyl}piperidin-1-yl)-2-methylnicotinate

Prepared according to the procedure described in Example 4(b) from 1-[3-cyano-5-(ethoxycarbonyl)-6-methylpyridin-2-yl]piperidine-4-carboxylic acid (31.7 mg, 0.1 mmol) and 1-(2-fluoro-5-methylphenyl)methanesulfonamide (20.3 mg, 0.1 mmol). Yield: 17.2 mg (34%).

¹H-NMR (400 MHz, DMSO-d₆) δ 1.29 (3H, t, J=7.1 Hz), 1.58-1.70 (2H, m), 1.82-1.90 (2H, m), 2.27 (3H, s), 2.5-2.65 (1H, m), 2.64 (3H, s), 3.10-3.20 (2H, m), 4.24 (2H, q, J=7.1 Hz), 4.49-4.57 (2H, m), 4.65 (2H, s), 7.09-7.17 (2H, m), 7.19-7.25 (1H, m), 8.33 (1H, s), 11.71 (1H, br. s, NH).

GTPγS(IC₅₀ μM): 0.035

### Example 8

### Ethyl 5-cyano-6-(4-{[(3-methoxybenzyl)sulfonyl]carbamoyl}piperidin-1-yl)-2-methylnicotinate

1-(3-methoxyphenyl)methanesulfonamide (125 mg, 0.62 mmol) was added after 1.5 hours to a stirred solution of 1-[3-cyano-5-(ethoxycarbonyl)-6-methylpyridin-2-yl]piperidine-4-carboxylic acid (125 mg, 0.39 mmol), TBTU (170 mg, 0.53 mmol) and DIPEA (0.15 ml, 0.86 mmol) in DCM (4 mL) and the reaction mixture was stirred at rt for 18 h. NaHCO₃(aq,sat) was added and the mixture was extracted with DCM (3 times). The combined organic layer was run through a phase separator and evaporated to give the crude product which was purified by preparative HPLC (Kromasil C8, 10 mm, using a gradient of CH₃CN/0.1 M NH₄OAc) to give the pure product after freeze drying. Yield: 143 mg (72%).

¹H NMR (500 MHz, DMSO-d₆): 1.31 (3H, t, J=7.1 Hz), 1.59-1.67 (2H, m), 1.80-1.86 (2H, m), 2.55-2.62 (1H, m), 2.65 (3H, s), 3.12-3.18 (2H, m), 3.75 (3H, s), 4.26 (2H, q, J=7.1 Hz), 4.51-4.56 (2H, m), 4.67 (2H, s), 6.85-6.87 (2H, m), 6.96-6.99 (1H, m), 7.30-7.34 (1H, m), 8.35 (1H, s), 11.61 (1H, s).

MS m/Z 501 (M+1), 499 (M−1).

GTPγS(IC₅₀ μM): 0.074

### Example 9

### Ethyl 5-cyano-6-(3-{[(2,6-difluorobenzyl)sulfonyl]carbamoyl}azetidin-1-yl)-2-ethylnicotinate

### (a) Ethyl 2-((dimethylamino)methylene)-3-oxopentanoate

1,1-Dimethoxy-N,N-dimethylmethanamine (5.09 mL, 42.0 mmol) was added drop-wise to ethyl 3-oxopentanoate (5.0 mL, 35.0 mmol) while stirring at r.t. The reaction mixture was stirred at r.t for 18 h and then was concentrated under reduced pressure and azeotroped with toluene (2×20 mL) producing ethyl 2-((dimethylamino)methylene)-3-oxopentanoate as an oil which was used without purification. Yield: 6.98 g (100%).

¹H NMR (400 MHz, CDCl₃): δ 1.10 (3H, t, J=7.7 Hz), 1.32 (3H, t, J=7.7 Hz), 2.67-2.69 (2H, m), 3.01 (6H, br s), 4.22 (2H, q, J=7.2 Hz), 7.64 (1H, s).

### (c) Ethyl 5-cyano-2-ethyl-6-oxo-1,6-dihydropyridine-3-carboxylate

NaH (60% dispersion in mineral oil, 1.54 g, 38.5 mmol) was added to a suspension of 2-cyanoacetamide (3.09 g, 36.8 mmol) in THF (100 mL). The mixture was stirred at r.t until gas evolution ceased, at which point ethyl 2-((dimethylamino)methylene)-3-oxopentanoate (6.98 g, 35.0 mmol) was added in one portion. The reaction mixture was stirred at r.t for 18 h and concentrated under reduced pressure to afford crude intermediate. The solids were dissolved in a minimum amount of warm water and then acidified to pH 1 with 5 M HCl. Filtration followed by drying under vacuum produced ethyl 5-cyano-2-ethyl-6-oxo-1,6-dihydropyridine-3-carboxylate as a solid. Yield: 6.28 g (81%).

¹H NMR (400 MHz, DMSO-d₆): δ 1.18 (3H, t, J=7.3 Hz), 1.29 (3H, t, J=7.0 Hz), 2.95 (2H, q, J=7.3 Hz), 4.24 (2H, q, J=7.0 Hz), 8.45 (1H, s), 12.79 (1H, br s).

MS m/Z: 221 (M+1).

### (d) Ethyl 6-chloro-5-cyano-2-ethylnicotinate

A suspension of ethyl 5-cyano-2-ethyl-6-oxo-1,6-dihydropyridine-3-carboxylate (6.28 g, 28.5 mmol) in POCl₃ (10.4 mL, 114 mmol) was heated to 100° C. for 6 h. The reaction mixture was poured onto ice and then basified with solid K₂CO₃. The aqueous phase was extracted with DCM (3×100 mL) and the organics dried (MgSO₄) and concentrated under reduced pressure to afford ethyl 6-chloro-5-cyano-2-ethylnicotinate as a solid, which was used without further purification. Yield: 6.17 g (91%).

¹H NMR (400 MHz, CDCl₃): δ 1.32 (3H, t, J=7.4 Hz), 1.42 (3H, t, J=7.4 Hz), 3.23 (2H, q, J=7.4 Hz), 4.42 (2H, q, J=7.4 Hz), 8.45 (1H, s).

MS m/Z: 239 (M+1).

### (e) 1-(3-Cyano-5-(ethoxycarbonyl)-6-ethylpyridin-2-yl)azetidine-3-carboxylic Acid

To a solution of ethyl 6-chloro-5-cyano-2-ethylnicotinate (0.290 g, 1.22 mmol) and DIPEA (0.635 mL, 3.65 mmol) in DMF (3.0 mL) was added azetidine-3-carboxylic acid (0.135 g, 1.34 mmol) and the resulting heterogenous mixture was heated to 90° C. for 3 h. The reaction mixture was diluted with EtOAc (75 mL), washed with saturated NH₄Cl (3×50 mL), brine (50 mL), dried (MgSO₄) and filtered through silica gel. Concentration followed by flash chromatography (1% HOAc, 20% EtOAc, hexanes) gave 1-(3-cyano-5-(ethoxycarbonyl)-6-ethylpyridin-2-yl)azetidine-3-carboxylic acid as a solid. Yield: 0.047 g (13%).

¹H NMR (400 MHz, CDCl₃): δ 1.22 (3H, t, J=7.4 Hz), 1.37 (3H, t, J=7.0 Hz), 3.10 (2H, q, J=7.4 Hz), 3.60-3.68 (1H, m), 4.31 (2H, q, J=7.4 Hz), 4.58-4.66 (4H, m), 8.27 (1H, s).

MS m/Z: 304 (M+1).

### (f) Ethyl 5-cyano-6-(3-{[(2,6-difluorobenzyl)sulfonyl]carbamoyl}azetidin-1-yl)-2-ethylnicotinate

1-(2,6-difluorophenyl)methanesulfonamide (22.8 mg, 0.11 mmol) was added after 10 minutes to a solution of 1-(3-cyano-5-(ethoxycarbonyl)-6-ethylpyridin-2-yl)azetidine-3-carboxylic acid (28.9 mg, 0.1 mmol), TBTU (160 mg, 0.5 mmol) and DIPEA (65 mg, 0.5 mmol) in DCM at r.t. and the mixture was stirred over night. 0.1 M KHSO₄(aq) (2 mL) was added and the organic phase was collected using a phase separator. Evaporation of the solvent gave a crude product which was purified by preparative HPLC (Preparative HPLC was performed using Waters Fraction Lynx Purification system with Kromasil C₈ 5 m 20×100 mm columns. The mobile phase used was a gradient of CH₃CN/0.1% HOAc (pH=4). The flow was 30 mL/minute. MS triggered fraction collection was used. Mass spectra was recorded on either a Micromass ZQ single quadrupole or a Micromass Quattro micro, both equipped with a pneumatically assisted electro spray interface.) to give the pure product after evaporation of the eluant. Yield: 48.6 mg (98%).

¹H NMR (300 MHz, DMSO-d₆) δ 1.18 (3H, t, J=7.4 Hz), 1.30 (3H, t, J=7.1 Hz), 3.02 (2H, q, J=7.4 Hz), 3.55-3.65 (1H, m), 4.24 (2H, q, J=7.1 Hz), 4.32-4.42 (2H, m), 4.42-4.53 (2H, m), 4.80 (2H, s), 7.11-7.25 (2H, m), 7.43-7.63 (1H, m), 8.31 (1H, s), 12.02-12.25 (1H, m)

MS m/Z: 493 (M+1), 491 (M−1)

GTPγS(IC₅₀ μM): 0.046

### Example 10

### Ethyl 5-cyano-2-ethyl-6-(3-{[(4-fluoro-3-methylbenzyl)sulfonyl]carbamoyl}azetidin-1-yl)nicotinate

Prepared according to Example 9(e) using 1-(3-cyano-5-(ethoxycarbonyl)-6-ethylpyridin-2-yl)azetidine-3-carboxylic acid (28.9 mg, 0.1 mmol) and 1-(4-fluoro-3-methylphenyl)methanesulfonamide (22.4 mg, 0.11 mmol) to give ethyl 5-cyano-2-ethyl-6-(3-{[(4-fluoro-3-methylbenzyl)sulfonyl]carbamoyl}azetidin-1-yl)nicotinate. Yield: 2 mg (4

¹H NMR (500 MHz, DMSO-d₆) δ 1.17 (3H, t, J=7.4 Hz), 1.30 (3H, t, J=7.0 Hz), 2.18 (3H, s), 3.01 (2H, q, J=7.4 Hz), 3.34-3.49 (1H, m), 4.19-4.33 (4H, m), 4.34-4.45 (2H, m), 4.46-4.58 (2H, m), 7.03-7.10 (1H, m), 7.11-7.23 (2H, m), 8.30 (1H, s), 11.75-11.86 (1H, m)

MS m/Z: 489 (M+1), 487 (M−1)

GTPγS(IC₅₀ μM): 0.064

### Example 11

### Ethyl 6-(3-{[(2-chloro-4-fluorobenzyl)sulfonyl]carbamoyl}azetidin-1-yl)-5-cyano-2-ethylnicotinate

Prepared according to Example 9(e) using 1-(3-cyano-5-(ethoxycarbonyl)-6-ethylpyridin-2-yl)azetidine-3-carboxylic acid (28.9 mg, 0.1 mmol) and 1-(2-chloro-4-fluorophenyl)methanesulfonamide (24.6 mg, 0.11 mmol) to give ethyl 6-(3-{[(2-chloro-4-fluorobenzyl)sulfonyl]carbamoyl}azetidin-1-yl)-5-cyano-2-ethylnicotinate. Yield: 49.6 mg (97%).

¹H NMR (400 MHz, DMSO-d₆) δ 1.17 (3H, t, J=7.4 Hz), 1.30 (3H, t, J=7.2 Hz), 3.01 (2H, q, J=7.5 Hz), 3.54-3.65 (1H, m), 4.24 (2H, q, J=7.1 Hz), 4.33-4.40 (2H, m), 4.41-4.49 (2H, m), 4.87 (2H, s), 7.23-7.35 (1H, m), 7.48-7.63 (2H, m), 8.30 (1H, s), 11.97-12.07 (1H, m)

MS m/Z: 509 (M+1), 507 (M−1)

GTPγS(IC₅₀ μM): 0.036

### Example 12

### Ethyl 5-cyano-6-(4-{[(5-fluoro-2-methylbenzyl)sulfonyl]carbamoyl}piperidin-1-yl)-2-methylnicotinate

Prepared according to Example 9(e) from 1-[3-cyano-5-(ethoxycarbonyl)-6-methylpyridin-2-yl]piperidine-4-carboxylic acid (31.7 mg, 0.1 mmol) and 1-(5-fluoro-2-methylphenyl)methanesulfonamide (22.4 mg, 0.11 mmol to give ethyl 5-cyano-6-(4-{[(5-fluoro-2-methylbenzyl)sulfonyl]carbamoyl}piperidin-1-yl)-2-methylnicotinate. Yield: 13.7 mg (27%).

¹H NMR (400 MHz, DMSO-d₆) δ 1.30 (3H, t, J=7.2 Hz), 1.58-1.72 (2H, m), 1.84-1.94 (2H, m), 2.34 (3H, s), 2.50-2.54 (1H, m), 2.65 (3H, s), 3.10-3.24 (2H, m), 4.25 (2H, q, J=7.1 Hz), 4.49-4.59 (2H, m), 4.74 (2H, s), 6.97-7.05 (1H, m), 7.08-7.18 (1H, m), 7.25-7.34 (1H, m), 8.34 (1H, s), 11.77-11.86 (1H, m)

MS m/Z: 503 (M+1), 501 (M−1)

GTPγS(IC₅₀ μM): 0.49

### Example 13

### Ethyl 5-cyano-2-methyl-6-(4-{[2,3,6-trifluorobenzyl)sulfonyl]carbamoyl}piperidin-1-yl)nicotinate

Prepared according to Example 9(e) from 1-[3-cyano-5-(ethoxycarbonyl)-6-methylpyridin-2-yl]piperidine-4-carboxylic acid (31.7 mg, 0.1 mmol) and 1-(2,3,6-trifluorophenyl)methanesulfonamide (22.5 mg, 0.1 mmol) to give ethyl 5-cyano-2-methyl-6-(4-{[(2,3,6-trifluorobenzyl)sulfonyl]carbamoyl}piperidin-1-yl)nicotinate. Yield: 6.4 mg (12%).

¹H NMR (400 MHz, DMSO-d₆) δ 1.30 (3H, t, J=7.1 Hz), 1.56-1.73 (2H, m), 1.85-1.96 (2H, m), 2.50-2.54 (1H, m), 2.65 (3H, s), 3.13-3.23 (2H, m), 4.25 (2H, q, J=7.1 Hz), 4.50-4.60 (2H, m), 4.80 (2H, s), 7.20-7.32 (1H, m), 7.53-7.68 (1H, m), 8.34 (1H, s), 11.94-12.00 (1H, m)

MS m/Z: 525 (M+1), 523 (M−1)

GTPγS(IC₅₀ μM): 0.071

### Example 14

### Ethyl 5-cyano-6-(4-{[(2,4-difluorobenzyl)sulfonyl]carbamoyl}piperidin-1-yl)-2-methylnicotinate

Prepared according to Example 9(e) from 1-[3-cyano-5-(ethoxycarbonyl)-6-methylpyridin-2-yl]piperidine-4-carboxylic acid (31.7 mg, 0.1 mmol) and 1-(2,4-difluorophenyl)methanesulfonamide (22.8 mg, 0.11 mmol) to give Ethyl 5-cyano-6-(4-{[(2,4-difluorobenzyl)sulfonyl]carbamoyl}piperidin-1-yl)-2-methylnicotinate. Yield: 3.9 mg (8%).

¹H NMR (400 MHz, DMSO-d₆) δ 1.30 (3H, t, J=7.1 Hz), 1.58-1.71 (2H, m), 1.84-1.92 (2H, m), 2.50-2.54 (1H, m), 2.65 (3H, s), 3.10-3.22 (2H, m), 4.25 (2H, q, J=7.1 Hz), 4.49-4.60 (2H, m), 4.73 (2H, s), 7.12-7.22 (1H, m), 7.28-7.39 (1H, m), 7.41-7.52 (1H, m), 8.34 (1H, s), 11.72-11.78 (1H, m)

MS m/Z: 507 (M+1), 505 (M−1)

GTPγS(IC₅₀ μM): 0.016

### Example 15

### Ethyl 5-cyano-6-(4-{[(2,6-difluorobenzyl)sulfonyl]carbamoyl}piperidin-1-yl)-2-ethylnicotinate

### (a) 1-[3-cyano-5-(ethoxycarbonyl)-6-ethylpyridin-2-yl]piperidine-4-carboxylic Acid

TEA (848 mg, 8.38 mml) was added to a solution of ethyl 6-chloro-5-cyano-2-ethylnicotinate (500 mg, 2.095 mmol) and piperidine-4-carboxylic acid (297.6 mg, 2.30 mmol) in EtOH (10 mL) and the mixture was heated to 120° C. for 10 minutes using microwave single node heating. The solvent was evaporated and the residue was dissolved in DCM. The organic phase was washed with 1% KHSO₄(aq). The water phase was extracted with DCM (2 times) and the combined organic phases was passed through a phase separator and evaporated to give a crude product which was purified by preparative HPLC (Kromasil C₈, 10 μm using a gradient of CH₃CN/0.2% HOAc) to give 1-[3-cyano-5-(ethoxycarbonyl)-6-ethylpyridin-2-yl]piperidine-4-carboxylic acid as a white solid. Yield: 574 mg (83%).

¹H-NMR (400 MHz, DMSO-d₆) δ 1.16 (3H, t, J=7.5 Hz), 1.29 (3H, t, J=7.1 Hz), 1.54-1.66 (2H, m), 1.90-1.98 (2H, m), 2.55-2.65 (1H, m), 3.02 (2H, q, J=7.5 Hz), 3.2-3.4 (2H, m, partly hidden by the water peak in DMSO), 4.24 (2H, q, J=7.1 Hz), 4.40-4.50 (2H, m), 8.31 (1H, s).

MS m/Z: 332 (M+1).

### (b) Ethyl 5-cyano-6-(4-{[(2,6-difluorobenzyl)sulfonyl]carbamoyl}piperidin-1-yl)-2-ethylnicotinate

Prepared according to Example 9(e) from 1-[3-cyano-5-(ethoxycarbonyl)-6-ethylpyridin-2-yl]piperidine-4-carboxylic acid (33.1 mg, 0.1 mmol) and 1-(2,6-difluorophenyl)methanesulfonamide (22.8 mg, 0.11 mmol) to give ethyl 5-cyano-6-(4-{[(2,6-difluorobenzyl)sulfonyl]carbamoyl}piperidin-1-yl)-2-ethylnicotinate. Yield: 18 mg (35%).

¹H NMR (500 MHz, DMSO-d₆) δ 1.18 (3H, t, J=7.4 Hz), 1.30 (3H, t, J=7.1 Hz), 1.57-1.71 (2H, m), 1.84-1.95 (2H, m), 2.46-2.53 (1H, m), 3.03 (2H, q, J=7.4 Hz), 3.14-3.22 (2H, m), 4.25 (2H, q, J=7.1 Hz), 4.51-4.60 (2H, m), 4.66-4.79 (2H, m), 7.12-7.24 (1H, m), 7.44-7.57 (2H, m), 8.33 (1H, s), 11.88-11.93 (1H, m)

MS m/Z: 521 (M+1), 519 (M−1)

GTPγS(IC₅₀ μM): 0.048

### Example 16

### Ethyl 6-(4-{[(2-chloro-4-fluorobenzyl)sulfonyl]carbamoyl}piperidin-1-yl)-5-cyano-2-ethylnicotinate

Prepared according to Example 9(e) from 1-[3-cyano-5-(ethoxycarbonyl)-6-ethylpyridin-2-yl]piperidine-4-carboxylic acid (33.1 mg, 0.1 mmol) and 1-(2-chloro-4-fluorophenyl)methanesulfonamide (24.6 mg) to give ethyl 6-(4-{[(2-chloro-4-fluorobenzyl)sulfonyl]carbamoyl}piperidin-1-yl)-5-cyano-2-ethylnicotinate. Yield: 12.2 mg (22%).

¹H NMR (500 MHz, DMSO-d₆) δ 1.19 (3H, t, J=7.4 Hz), 1.30 (3H, t, J=7.0 Hz), 1.58-1.72 (2H, m), 1.85-1.94 (2H, m), 2.55-2.63 (1H, m), 3.04 (2H, t, J=11.1 Hz), 3.18 (2H, m), 4.23-4.27 (2H, m), 4.51-4.62 (2H, m), 4.83 (2H, s), 7.27-7.37 (1H, m), 7.45-7.60 (2H, m), 8.34 (1H, s), 11.78-11.86 (1H, m)

MS m/Z: 537 (M+1), 535 (M−1)

GTPγS(IC₅₀ μM): 0.211

### Example 17

### Ethyl 5-cyano-2-ethyl-6-(4-{[(2,3,6-trifluorobenzyl)sulfonyl]carbamoyl}piperidin-1-yl)nicotinate

Prepared according to Example 9(e) from 1-[3-cyano-5-(ethoxycarbonyl)-6-ethylpyridin-2-yl]piperidine-4-carboxylic acid (33.1 mg, 0.1 mmol) and 1-(2,3,6-trifluorophenyl)methanesulfonamide (22.5 mg, 0.1 mmol) to give ethyl 5-cyano-2-ethyl-6-(4-{[(2,3,6-trifluorobenzyl)sulfonyl]carbamoyl}piperidin-1-yl)nicotinate. Yield: 10.8 mg (20%).

¹H NMR (500 MHz, DMSO-d₆) δ 1.19 (3H, t, J=7.4 Hz), 1.30 (3H, t, J=7.0 Hz), 1.57-1.72 (2H, m), 1.84-1.96 (2H, m), 2.56-2.68 (1H, m), 3.04 (2H, q, J=7.4 Hz), 3.14-3.24 (2H, m), 4.25 (2H, q, J=7.1 Hz), 4.50-4.62 (2H, m), 4.80 (2H, s), 7.22-7.30 (1H, m), 7.55-7.67 (1H, m), 8.34 (1H, s), 11.93-12.01 (1H, m)

MS m/Z: 539 (M+1), 537 (M−1)

GTPγS(IC₅₀ μM): 0.097

### Example 18

### Ethyl 5-cyano-6-(4-{[(2,4-difluorobenzyl)sulfonyl]carbamoyl}piperidin-1-yl)-2-ethylnicotinate

Prepared according to Example 9(e) from 1-[3-cyano-5-(ethoxycarbonyl)-6-ethylpyridin-2-yl]piperidine-4-carboxylic acid (33.1 mg, 0.1 mmol) and 1-(2,4-difluorophenyl)methanesulfonamide (22.8 mg 0.11 mmol) to give ethyl 5-cyano-6-(4-{[(2,4-difluorobenzyl)sulfonyl]carbamoyl}piperidin-1-yl)-2-ethylnicotinate. Yield: 17.6 mg (34%).

¹H NMR (400 MHz, DMSO-d₆) δ 1.19 (3H, t, J=7.5 Hz), 1.30 (3H, t, J=7.1 Hz), 1.57-1.72 (2H, m), 1.82-1.96 (2H, m), 2.54-2.67 (1H, m), 3.04 (2H, q, J=7.5 Hz), 3.11-3.24 (2H, m), 4.25 (2H, q, J=7.1 Hz), 4.56 (2H, d, J=42.8 Hz), 4.72 (2H, s), 7.13-7.21 (1H, m), 7.28-7.38 (1H, m), 7.41-7.52 (1H, m), 8.34 (1H, s), 11.70-11.83 (1H, m)

MS m/Z: 521 (M+1), 519 (M−1)

GTPγS(IC₅₀ μM): 0.02

### Example 19

### Ethyl 6-(4-[{(4-chloro-2-fluorobenzyl)sulfonyl]carbamoyl}piperidin-1-yl)-5-cyano-2-ethylnicotinate

Prepared according to Example 9(e) from 1-[3-cyano-5-(ethoxycarbonyl)-6-ethylpyridin-2-yl]piperidine-4-carboxylic acid (33.1 mg, 0.1 mmol) and 1-(4-chloro-2-fluorophenyl)methanesulfonamide (24.6 mg, 0.11 mmol) to give Ethyl 6-(4-{[(4-chloro-2-fluorobenzyl)sulfonyl]carbamoyl}piperidin-1-yl)-5-cyano-2-ethylnicotinate. Yield: 10.9 mg (20%).

¹H NMR (500 MHz, DMSO-d₆) δ 1.19 (3H, t, J=7.4 Hz), 1.30 (3H, t, J=7.1 Hz), 1.57-1.72 (2H, m), 1.85-1.95 (2H, m), 2.58-2.68 (1H, m), 3.02-3.06 (2H, m), 3.13-3.23 (2H, m), 4.25 (2H, q, J=7.1 Hz), 4.52-4.62 (2H, m), 4.83 (2H, s), 7.28-7.36 (1H, m), 7.47-7.57 (2H, m), 8.34 (1H, s), 11.78-11.86 (1H, m)

MS m/Z: 521 (M+1), 519 (M−1)

GTPγS(IC₅₀ μM): 0.01

### Example 20

### Ethyl 5-cyano-6-(3-{[(2,4-difluorobenzyl)sulfonyl]carbamoyl}azetidin-1-yl)-2-methylnicotinate

1-(2,4-difluorophenyl)methanesulfonamide (20.7 mg, 0.1 mmol) was added after 10 minutes to a solution of 1-(3-cyano-5-(ethoxycarbonyl)-6-methylpyridin-2-yl)azetidine-3-carboxylic acid (28.9 mg, 0.1 mmol), PyBrop (139.9 mg, 0.3 mmol) and DIPEA (129.3 mg, 1.0 mmol) in DCM at r.t. and the mixture was stirred over night. 0.1 M KHSO₄(aq) (2 mL) was added and the organic phase was collected using a phase separator. Evaporation of the solvent gave a crude product which was purified by preparative HPLC (Preparative HPLC was performed using waters Fraction Prep 3000 System with Kromasil C₈, 10 μm, 20×250 mm column. The mobile phase used was a gradient of CH₃CN/0.1% HOAc in water and the flow was 30 mL/minute.) to give ethyl 5-cyano-6-(3-{[(2,4-difluorobenzyl)sulfonyl]carbamoyl}azetidin-1-yl)-2-methylnicotinate. Yield: 17.9 mg (37

¹H NMR (300 MHz, DMSO-d₆) δ 1.30 (3H, t, J=7.1 Hz), 2.64 (3H, s), 3.48-3.68 (1H, m), 4.24 (2H, q, J=7.0 Hz), 4.29-4.40 (2H, m), 4.40-4.52 (2H, m), 4.77 (2H, s), 7.09-7.21 (1H, m), 7.24-7.37 (1H, m), 7.45-7.59 (1H, m), 8.31 (1H, s), 11.92-12.04 (1H, m)

MS m/Z: 479 (M+1), 477 (M−1)

GTPγS(IC₅₀ μM): 0.035

### Example 21

### Ethyl 5-cyano-6-(3-{[(2,4-difluorobenzyl)sulfonyl]carbamoyl}azetidin-1-yl)-2-ethylnicotinate

Prepared according to Example 20 from 1-(3-cyano-5-(ethoxycarbonyl)-6-ethylpyridin-2-yl)azetidine-3-carboxylic acid (30.3 mg, 0.1 mmol) and 1-(2,4-difluorophenyl)methanesulfonamide (20.7 mg, 0.1 mmol) to give ethyl 5-cyano-6-(3-{[(2,4-difluorobenzyl)sulfonyl]carbamoyl}azetidin-1-yl)-2-ethylnicotinate. Yield: 27.2 mg (55%).

¹H NMR (300 MHz, DMSO-d₆) δ 1.17 (3H, t, J=7.4 Hz), 1.30 (3H, t, J=7.1 Hz), 3.02 (2H, q, J=7.3 Hz), 3.52-3.66 (1H, m), 4.24 (2H, q, J=6.9 Hz), 4.30-4.40 (2H, m), 4.40-4.52 (2H, m), 4.78 (2H, s), 7.10-7.21 (1H, m), 7.24-7.35 (1H, m), 7.46-7.59 (1H, m), 8.30 (1H, s), 11.93-12.06 (1H, m)

MS m/Z: 493 (M+1), 491 (M−1)

GTPγS(IC₅₀ μM): 0.03

### Example 22

### Ethyl 5-cyano-2-ethyl-6-(3-{[(4-fluorobenzyl)sulfonyl]carbamoyl}azetidin-1-yl)nicotinate

Prepared according to Example 20 from 1-(3-cyano-5-(ethoxycarbonyl)-6-ethylpyridin-2-yl)azetidine-3-carboxylic acid (30.3 mg, 0.1 mmol) and 1-(4-fluorophenyl)methanesulfonamide (18.9 mg, 0.1 mmol) to give ethyl 5-cyano-2-ethyl-6-(3-{[(4-fluorobenzyl)sulfonyl]carbamoyl}azetidin-1-yl)nicotinate. Yield: 25.4 mg (53%).

¹H NMR (300 MHz, DMSO-d₆) δ 1.18 (3H, t, J=7.4 Hz), 1.30 (3H, t, J=7.1 Hz), 3.02 (2H, q, J=7.4 Hz), 3.51-3.61 (1H, m), 4.24 (2H, q, J=7.1 Hz), 4.26-4.36 (2H, m), 4.37-4.48 (2H, m), 4.75 (2H, s), 7.13-7.27 (2H, m), 7.33-7.48 (2H, m), 8.30 (1H, s), 11.73-11.93 (1H, m)

MS m/Z: 475 (M+1), 473 (M−1)

GTPγS(IC₅₀ μM): 0.041

### Example 23

### Ethyl 6-(3-{[(4-chlorobenzyl)sulfonyl]carbamoyl}azetidin-1-yl)-5-cyano-2-ethylnicotinate

Prepared according to Example 20 from 1-(3-cyano-5-(ethoxycarbonyl)-6-ethylpyridin-2-yl)azetidine-3-carboxylic acid (30.3 mg, 0.1 mmol) and 1-(4-chlorophenyl)methanesulfonamide (20.6 mg, 0.1 mmol) to give ethyl 6-(3-{[(4-chlorobenzyl)sulfonyl]carbamoyl}azetidin-1-yl)-5-cyano-2-ethylnicotinate. Yield: 18.6 mg (38%).

¹H NMR (300 MHz, DMSO-d₆) δ 1.18 (3H, t, J=7.5 Hz), 1.30 (3H, t, J=7.1 Hz), 3.02 (2H, q, J=7.3 Hz), 3.48-3.65 (1H, m), 4.24 (2H, q, J=7.1 Hz), 4.26-4.36 (2H, m), 4.36-4.49 (2H, m), 4.75 (2H, s), 7.36 (2H, d, J=8.6 Hz), 7.44 (2H, d, J=8.4 Hz), 8.30 (1H, s), 11.76-11.92 (1H, m)

MS m/Z: 491 (M+1), 489 (M−1)

GTPγS(IC₅₀ μM): 0.022

### Example 24

### Ethyl 5-cyano-2-ethyl-6-(4-{[(4-fluorobenzyl)sulfonyl]carbamoyl}piperidin-1-yl)nicotinate

Prepared according to Example 20 from 1-[3-cyano-5-(ethoxycarbonyl)-6-ethylpyridin-2-yl]piperidine-4-carboxylic (33.1 mg, 0.1 mmol) and 1-(4-fluorophenyl)methanesulfonamide (18.9 mg, 0.1 mmol) to give ethyl 5-cyano-2-ethyl-6-(4-{[(4-fluorobenzyl)sulfonyl]carbamoyl}piperidin-1-yl)nicotinate. Yield: 10.2 mg (20%).

¹H NMR (300 MHz, DMSO-d₆) δ 1.19 (3H, t, J=7.4 Hz), 1.31 (3H, t, J=7.1 Hz), 1.53-1.73 (2H, m), 1.78-1.93 (2H, m), 2.38-2.54 (1H, m), 3.04 (2H, q, J=7.4 Hz), 3.08-3.25 (2H, m), 4.25 (2H, q, J=7.1 Hz), 4.49-4.61 (2H, m), 4.68 (2H, s), 7.19-7.28 (2H, m), 7.29-7.37 (2H, m), 8.33 (1H, s), 11.58-11.67 (1H, m)

MS m/Z: 503 (M+1), 501 (M−1)

GTPγS(IC₅₀ μM): 0.036

### Example 25

### Ethyl 6-(4-{[(4-chlorobenzyl)sulfonyl]carbamoyl}piperidin-1-yl)-5-cyano-2-ethylnicotinate

Prepared according to Example 20 from 1-[3-cyano-5-(ethoxycarbonyl)-6-ethylpyridin-2-yl]piperidine-4-carboxylic (33.1 mg, 0.1 mmol) and 1-(4-chlorophenyl)methanesulfonamide (20.6 mg, 0.1 mmol) to give Ethyl 6-(4-{[(4-chlorobenzyl)sulfonyl]carbamoyl}piperidin-1-yl)-5-cyano-2-ethylnicotinate. Yield: 15.6 mg (30%).

¹H NMR (300 MHz, DMSO-d₆) δ 1.19 (3H, t, J=7.4 Hz), 1.31 (3H, t, J=7.1 Hz), 1.55-1.73 (2H, m), 1.78-1.91 (2H, m), 2.43-2.58 (1H, m), 3.04 (2H, q, J=7.4 Hz), 3.10-3.22 (2H, m), 4.25 (2H, q, J=7.1 Hz), 4.49-4.61 (2H, m), 4.69 (2H, s), 7.30 (2H, d, J=8.4 Hz), 7.47 (2H, d, J=8.4 Hz), 8.33 (1H, s), 11.59-11.66 (1H, m)

MS m/Z: 519 (M+1), 517 (M−1)

GTPγS(IC₅₀ μM): 0.04

### Example 26

### Ethyl 5-cyano-6-(3-{[(2-fluorobenzyl)sulfonyl]carbamoyl}azetidin-1-yl)-2-methylnicotinate

1-(2-fluorophenyl)methanesulfonamide (22.7 mg, 0.12 mmol) was added after 10 minutes to a solution of 1-(3-cyano-5-(ethoxycarbonyl)-6-methylpyridin-2-yl)azetidine-3-carboxylic acid (28.9 mg, 0.1 mmol), PyBrop (70 mg, 0.15 mmol) and DIPEA (129.3 mg, 1.0 mmol) in DCM at r.t. and the mixture was stirred over night. 0.1 M KHSO₄(aq) (2 mL) was added and the organic phase was collected using a phase separator. Evaporation of the solvent gave a crude product which was purified by using Waters Oasis MAX cartridges (2×500 mg, tetra alkyl ammonium phase). Addition of product mixture at pH about 10 (titration with 0.1 M NaOH), wash with additional 0.1 M NaOH (2 mL), the phosphineoxide was eluted with CH₃CN/water 1/1 (4.5 mL) and 100% CH₃CN. Product eluted with 90% CH₃CN and 2% Formic acid. Evaporation of the solvents gave the pure product. Yield: 23 mg (50%).

¹H NMR (400 MHz, DMSO-d₆) δ 1.30 (3H, t, J=7.1 Hz), 2.64 (3H, s), 3.52-3.64 (1H, m), 4.24 (2H, q, J=7.1 Hz), 4.32-4.39 (2H, m), 4.40-4.49 (2H, m), 4.79 (2H, s), 7.18-7.29 (2H, m), 7.40-7.52 (2H, m), 8.32 (1H, s), 11.94-12.00 (1H, m)

MS m/Z: 461 (M+1), 459 (M−1)

GTPγS(IC₅₀ μM): 0.035

### Example 27

### Ethyl 5-cyano-6-(4-{[(2-fluorobenzyl)sulfonyl]carbamoyl}piperidin-1-yl)-2-methylnicotinate

Prepared essentially according to Example 26 from 1-[3-cyano-5-(ethoxycarbonyl)-6-methylpyridin-2-yl]piperidine-4-carboxylic acid (31.7 mg, 0.1 mmol) and 1-(2-fluorophenyl)methanesulfonamide (22.7 mg, 0.12 mmol) to give ethyl 5-cyano-6-(4-{[(2-fluorobenzyl)sulfonyl]carbamoyl}piperidin-1-yl)-2-methylnicotinate. Yield: 22 mg (45%).

¹H NMR (400 MHz, DMSO-d₆) δ 1.31 (3H, t, J=7.1 Hz), 1.58-1.73 (2H, m), 1.84-1.94 (2H, m), 2.59-2.63 (1H, m), 2.65 (3H, s), 3.09-3.22 (2H, m), 4.25 (2H, q, J=7.1 Hz), 4.49-4.61 (2H, m), 4.75 (2H, s), 7.22-7.31 (2H, m), 7.37-7.51 (2H, m), 8.34 (1H, s), 11.72-11.77 (1H, m)

MS m/Z: 489 (M+1), 487 (M−1)

GTPγS(IC₅₀ μM): 0.026

### Example 28

### Ethyl 6-{3-[3-(benzyloxy)-3-oxopropyl]-4-[(benzylsulfonyl)carbamoyl]piperidin-1-yl}-5-cyano-2-methylnicotinate

### (a) tert-Butyl 4-pyrrolidin-1-yl-3,6-dihydropyridine-1(2H)-carboxylate

Pyrrolidine (5 mL, 60.5 mmol) was added to a solution of tert-butyl 4-oxopiperidine-1-carboxylate (11.3 g, 56.7 mmol) and p-TsOH (57 mg, 0.16 mmol) in dry toluene (75 mL) and the reaction mixture was heated during 20 hours using a Deans & Stark trap remove the water liberated in the reaction. Evaporation of the solvent gave the desired product which was used without further purification in the next step. Yield: 14.9 g (104%).

### (b) tert-Butyl 3-[3-(benzyloxy)-3-oxopropyl]-4-oxopiperidine-1-carboxylate

Benzyl acrylate (10.0 g, 61.9 mmol) was added to a solution of tert-butyl 3-[3-(benzyloxy)-3-oxopropyl]-4-oxopiperidine-1-carboxylate (14.9 g, 56.7 mmol) in dry toluene (55 mL). The reaction mixture was heated to reflux for three days. Water (25 mL) was added and the mixture was heated to reflux for another 2 hours. Additional water was added and the organic layer was separated and the aqueous layer was extracted with toluene (2 times). The combined organic layer was dried (MgSO₄), filtered and evaporated to give the crude product which was used without further purification in the next step. Yield: 18.7 g (91%).

MS m/Z: 362 (M+1).

### (b) tert-Butyl-3-[3-(benzyloxy)-3-oxopropyl]-4-(methoxymethylene)piperidine-1-carboxylate

n-BuLi (3 mL, 1.6 M in hexane, 4.8 mmol) was added to a suspension of (methoxymethyl)(triphenyl)phosphonium chloride (1.26 g, 3.68 mmol) in dry THF (20 mL) at r.t. The solution turned red and the slurry was stirred at r.t for 25 minutes. An additional amount of n-BuLi (1.3 mL, 1.6 M in hexane, 2.08 mmol) was added and the mixture became homogenous. The crude tert-butyl 3-[3-(benzyloxy)-3-oxopropyl]-4-oxopiperidine-1-carboxylate (997 mg, 2.76 mmol) from above dissolved in dry THF (5 mL) was added to the red solution of the phosphonium ylide at r.t. The reaction mixture was stirred at r.t for 2 hours and quenched with water, the THF was evaporated and the remainings was extracted with diethyl ether (3 times). The combined organic layer was run through a phase separator and evaporated to give the crude product (1.82 g). The crude product was purified by filtration through a plug of Silica gel using Hexane/EtOAc (10:1) as eluent to give the desired product. Yield: 483 mg (45%).

MS m/Z: 390 (M+1)

### (d) Benzyl 3-(4-formylpiperidin-3-yl)propanoate

TFA (4 mL) was added to a solution of tert-butyl-3-[3-(benzyloxy)-3-oxopropyl]-4-(methoxymethylene)piperidine-1-carboxylate (480 mg, 1.23 mmol) in DCM (6 mL) and the reaction mixture was stirred at r.t for 3 hours. The solvent and excess TFA were evaporated to give the crude product which was used in the next step without further purification. Yield: 361 mg (106%).

MS m/Z: 276 (M+1).

### (e) Ethyl 6-{3-[3-(benzyloxy)-3-oxopropyl]-4-formylpiperidin-1-yl}-5-cyano-2-methylnicotinate

A microwave vial was charged with the crude benzyl 3-(4-formylpiperidin-3-yl)propanoate (360 mg, 1.31 mmol), ethyl 6-chloro-5-cyano-2-methylnicotinate (305 mg, 1.31 mmol), DIPEA (1.3 mL, 7.46 mmol), EtOH (6 mL) and water (2 mL) and heated to 120° C. for 5 minutes using microwave single node heating. NH₄Cl(aq) was added and the mixture was extracted with DCM (3 times). The combined organic layer was run through a phase separator and evaporated to give the crude product which was used without further purification in the next step. Yield: 760 mg (94%, purity 75%).

MS m/Z: 464 (M+1), 462 (M−1).

### (f) 3-[3-(Benzyloxy)-3-oxopropyl]-1-[3-cyano-5-(ethoxycarbonyl)-6-methylpyridin-2-yl]piperidine-4-carboxylic acid

NaH₂PO₄×H₂O (380 mg, 2.75 mmol) was added to a solution of ethyl 6-{3-[3-(benzyloxy)-3-oxopropyl]-4-formylpiperidin-1-yl}-5-cyano-2-methylnicotinate (760 mg, 75% pure, 1.23 mmol) in t-BuOH (10 mL) and water (2 mL) followed by NaClO₂ (228 mg, 2.52 mmol) and the reaction mixture was stirred at r.t for 1 hour. The organic solvent was evaporated and the acidic (pH 4) aqueous phase was extracted with DCM (3 times). The combined organic layer was run through a phase separator and evaporated to give the crude product which was used without further purification. Yield: 572 mg (97%).

### (g) Ethyl 6-{3-[3-(benzyloxy)-3-oxopropyl]-4-[(benzylsulfonyl)carbamoyl]piperidin-1-yl}-5-cyano-2-methylnicotinate

3-[3-(Benzyloxy)-3-oxopropyl]-1-[3-cyano-5-(ethoxycarbonyl)-6-methylpyridin-2-yl]piperidine-4-carboxylic acid (285 mg, 90% pure, 0.53 mmol), TBTU (227 mg, 0.71 mmol) and DIPEA (0.5 mL, 2.87 mmol) were suspended in dry DCM (4 mL) and stirred at r.t for 1.5 hours after which 1-phenylmethanesulfonamide (92 mg, 0.54 mmol) was added. The reaction mixture was stirred at r.t for 20 hours. NaHCO₃(aq) was added and the mixture was extracted with DCM (3 times). The combined organic layer was run through a phase separator and evaporated to give a crude product that was purified by preparative HPLC (Kromasil C₈ 10 μm, 21.5×250 mm using a gradient of CH₃CN/0.1 M NH₄OAc(aq) (25% CH₃CN to 60% CH₃CN)) to give the pure product. Yield: 60 mg (18

MS m/Z: 633 (M+1), 631 (M−1).

GTPγS(IC₅₀ μM): 0.442

### Example 29

### Ethyl 6-(3-[3-(benzyloxy)-3-oxopropyl]-4-{[(2,4-difluorobenzyl)sulfonyl]carbamoyl}piperidin-1-yl)-5-cyano-2-methylnicotinate

Prepared according to Example 28(g) from 3-[3-(Benzyloxy)-3-oxopropyl]-1-[3-cyano-5-(ethoxycarbonyl)-6-methylpyridin-2-yl]piperidine-4-carboxylic acid (285 mg, 90% pure, 0.53 mmol) and 1-(2,4-difluorophenyl)methanesulfonamide (93 mg, 0.45 mmol). Yield: 9.5 mg (3%).

MS m/Z: 669 (M+1), 667 (M−1).

GTPγS(IC₅₀ μM): 0.382

### Example 30

### 3-{4-[(Benzylsulfonyl)carbamoyl]-1-[3-cyano-5-(ethoxycarbonyl)-6-methylpyridin-2-yl]piperidin-3-yl}propanoic Acid

Ethyl 6-{3-[3-(benzyloxy)-3-oxopropyl]-4-[(benzylsulfonyl)carbamoyl]piperidin-1-yl}-5-cyano-2-methylnicotinate (137 mg, 0.22 mmol) was dissolved in MeOH (10 mL), Pd(OH)₂ (28.6, 0.041 mmol) and NH4COOH (194 mg, 3.1 mmol) were added. The reaction mixture was heated to 120° C. for 10 min in a single node microwave oven. LCMS showed 8% product. More NH4COOH and Pd(OH)2 were added and the reaction mixture was heated to 120° C. for 10 min in a single node microwave oven. This was repeated until no starting material was left (4 times). The mixture was filtered through a plug of Celite and the filtrate was evaporated. The residue was redissolved in DCM and NaHCO3(aq) was added. No phase separation was obtained so AcOH was added until pH 5. The organic layer was separated and the aqueous layer was extracted with DCM(×2). The combined organics was run through a phase separator and evaporated. The crude product was purified by purification method A (See General Experimental Procedure) to give the product as a solid. Yield: 43 mg (36%)

MS m/Z: 543 (M+1), 541 (M−1).

GTPγS(IC₅₀ μM): 0.057

1. A compound of formula I or a pharmaceutically acceptable salt thereof: 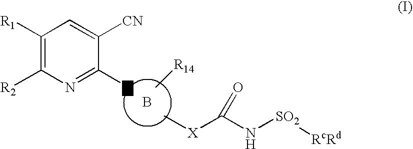
wherein
R₁ represents R₆OC(O);
R₂ represents methyl, ethyl or methylamino;
R₆ represents ethyl or halogenated ethyl;
R₁₄ represents H, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl;
R^{c} represents methylene (—CH₂—) or ethylene (—CH₂—CH₂—);
R^{d} represents phenyl, wherein the phenyl group optionally; is
substituted by one or more fluorine atom(s), and/or
substituted by one or more chlorine atom(s) only in one or more of the 2, 4, 5, 6-positions of the phenyl ring, and/or
mono-, tri-, tetra- or penta-substituted by methyl group(s), and/or
substituted by one or more methoxy group(s) only in one or more of the 2, 3, 5, 6-positions of the phenyl ring;
X represents a single bond or methylene (—CH₂—), wherein the methylene group may optionally be substituted with (C₁-C₆) alkyl; or
X represents a group (—CH₂—)n wherein n=2-6, which optionally is unsaturated and/or substituted by one or more substituent selected from halogen, hydroxyl or (C₁-C₆)alkyl;
B is a ring/ring system comprising a nitrogen which nitrogen is connected to the pyridine-ring (according to formula I) and the B-ring/ring system is connected to X in another of its positions, wherein the substituent R₁₄ is connected to the B ring/ring system in such a way that no quarternary ammonium compounds are formed (by this connection), and wherein the B ring/ring system is selected from 3-azetidin-1-ylene, 3-pyrrolidine-1-ylene and 4-piperidine-1-ylene; and
wherein the following compounds are not included:
ethyl 6-[3-({[(2-chlorobenzyl)sulfonyl]amino}carbonyl)azetidin-1-yl]-5-cyano-2-methylnicotinate
ethyl 6-[3-({[(4-chlorobenzyl)sulfonyl]amino}carbonyl)azetidin-1-yl]-5-cyano-2-methylnicotinate
ethyl 5-cyano-6-[3-({[(3-fluorobenzyl)sulfonyl]amino}carbonyl)azetidin-1-yl]-2-methylnicotinate
ethyl 5-cyano-2-methyl-6-[3-({[(2-phenylethyl)sulfonyl]amino}carbonyl)azetidin-1-yl]nicotinate
ethyl 5-cyano-2-methyl-6-[3-({[(2-methylbenzyl)sulfonyl]amino}carbonyl)azetidin-1-yl]nicotinate
ethyl 5-cyano-6-{3-[({[2-(4-fluorophenyl)ethyl]sulfonyl}amino)carbonyl]azetidin-1-yl}-2-methylnicotinate
ethyl 5-cyano-6-[3-({[(4-fluorobenzyl)sulfonyl]amino}carbonyl)azetidin-1-yl]-2-methylnicotinate
ethyl 5-cyano-6-{3-[({[2-(2-fluorophenyl)ethyl]sulfonyl}amino)carbonyl]azetidin-1-yl}-2-methylnicotinate
ethyl 5-cyano-6-{3-[({[2-(3-fluorophenyl)ethyl]sulfonyl}amino)carbonyl]azetidin-1-yl}-2-methylnicotinate
ethyl 6-(4-{[(benzylsulfonyl)amino]carbonyl}-4-methylpiperidin-1-yl)-5-cyano-2-methylnicotinate
ethyl 6-(4-{[(benzylsulfonyl)amino]carbonyl}piperidin-1-yl)-5-cyano-2-ethylnicotinate
ethyl 6-[4-({[(4-chlorobenzyl)sulfonyl]amino}carbonyl)piperidin-1-yl]-5-cyano-2-methylnicotinate
ethyl 6-(4-{[(benzylsulfonyl)amino]carbonyl}piperidin-1-yl)-5-cyano-2-methylnicotinate
ethyl 6-(3-{[(benzylsulfonyl)amino]carbonyl}azetidin-1-yl)-5-cyano-2-methylnicotinate
ethyl 5-cyano-2-methyl-6-[3-({[(3-methylbenzyl)sulfonyl]amino}carbonyl)azetidin-1-yl]nicotinate
ethyl 5-cyano-2-methyl-6-[3-({[(4-methylbenzyl)sulfonyl]amino}carbonyl)azetidin-1-yl]nicotinate
ethyl 5-cyano-6-[4-({[(4-fluorobenzyl)sulfonyl]amino}carbonyl)piperidin-1-yl]-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
ethyl 6-(3-{[(benzylsulfonyl)amino]carbonyl}azetidin-1-yl)-5-cyano-2-ethylnicotinate
ethyl 5-cyano-2-methyl-6-[4-({[(4-methylbenzyl)sulfonyl]amino}carbonyl)piperidin-1-yl]nicotinate
ethyl 5-cyano-6-[4-({[(3-fluorobenzyl)sulfonyl]amino}carbonyl)piperidin-1-yl]-2-methylnicotinate
ethyl 5-cyano-2-methyl-6-[4-({[(2-phenylethyl)sulfonyl]amino}carbonyl)piperidin-1-yl]nicotinate
ethyl 6-(3-{2-[(benzylsulfonyl)amino]-2-oxoethyl}azetidin-1-yl)-5-cyano-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-2-methyl-6-[4-({[(3-methylbenzyl)sulfonyl]amino}carbonyl)piperidin-1-yl]nicotinate and
ethyl 5-cyano-6-[4-({[(3,4-difluorobenzyl)sulfonyl]amino}carbonyl)piperidin-1-yl]-2-methylnicotinate.

2. A compound according to claim 1 wherein
R₆ represents ethyl or fluorinated ethyl; and
R₁₄ represents H, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl.

3. A compound according to claim 2 wherein:
X represents a single bond or methylene (—CH₂—).

4. A compound according to claim 3 wherein:
R₆ represents ethyl or 2,2,2-trifluoroethyl; and
R^{c} represents methylene (—CH₂—).

5. A compound according to claim 1 which is of the formula (Ia):

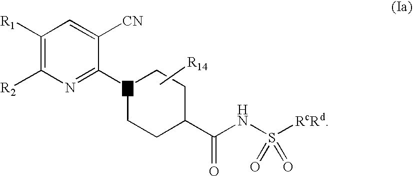

6. A compound according to claim 1 which is of the formula (Ib):

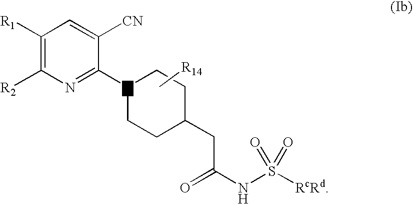

7. A compound according to claim 1 which is of the formula (Ic):

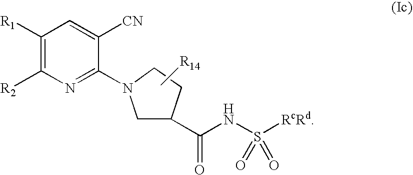

8. A compound according to claim 1 which is of the formula (Id):

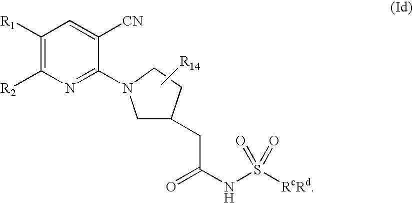

9. A compound according to claim 1 which is of the formula (Ie):

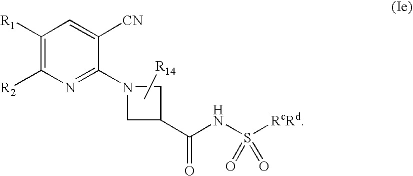

10. A compound according to claim 1 which is of the formula (If):

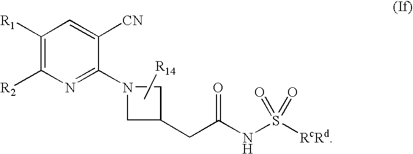

11. A compound according to claim 1 wherein R^{c} is methylene (—CH₂—).

12. A compound according to claim 1 wherein R^{c} is ethylene (—CH₂—CH₂—).

13. A compound according to claim 11 which is of the formula (Iaa):

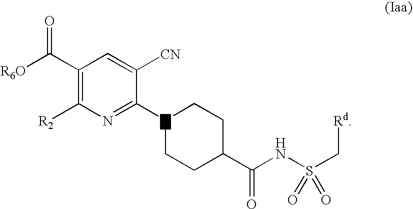

14. A compound according to claim 11 which is of the formula (Iab):

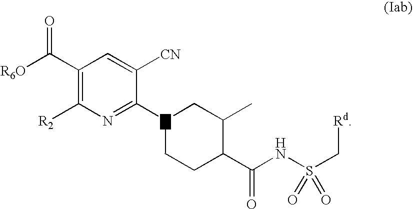

15. A compound according to claim 11 which is of the formula (Iac):

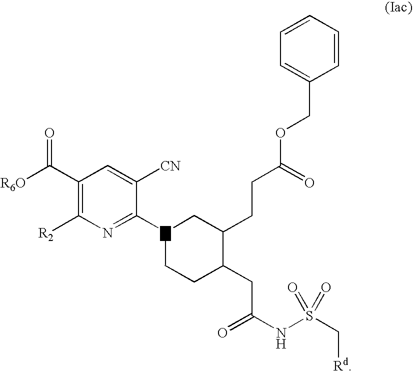

16. A compound according to claim 11 which is of the formula (Iad):

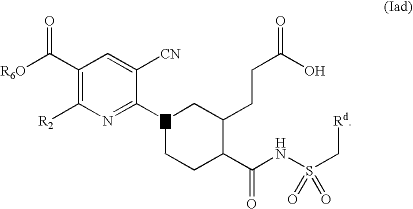

17. A compound according to claim 11 which is of the formula (Idd):

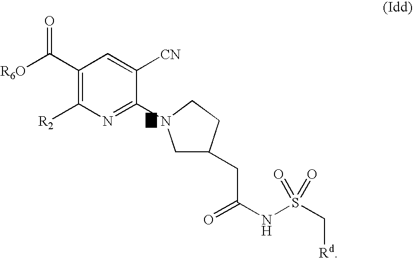

18. A compound according to claim 12 which is of the formula (Ide)

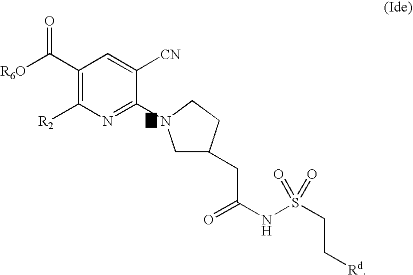

19. A compound according to claim 11 which is of the formula (Iee):

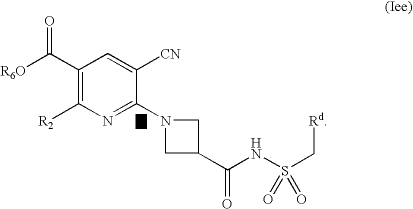

20. A compound selected from:
ethyl 6-(3-{2-[(benzylsulfonyl)amino]-2-oxoethyl}pyrrolidin-1-yl)-5-cyano-2-methylnicotinate
ethyl 5-cyano-2-methyl-6-[3-(2-oxo-2-{[(2-phenylethyl)sulfonyl]amino}ethyl)pyrrolidin-1-yl]nicotinate
2,2,2-trifluoroethyl 5-cyano-6-(4-{[(4-fluorobenzyl)sulfonyl]carbamoyl}piperidin-1-yl)-2-methylnicotinate
ethyl 5-cyano-6-(3-{[(2-fluoro-5-methylbenzyl)sulfonyl]carbamoyl}azetidin-1-yl)-2-methylnicotinate
ethyl 6-{4-[(benzylsulfonyl)carbamoyl]-3-methylpiperidin-1-yl}-5-cyano-2-methylnicotinate
ethyl 6-{4-[(benzylsulfonyl)carbamoyl]piperidin-1-yl}-5-cyano-2-(methylamino)nicotinate
ethyl 5-cyano-6-(4-{[(2-fluoro-5-methylbenzyl)sulfonyl]carbamoyl}piperidin-1-yl)-2-methylnicotinate
ethyl 5-cyano-6-(4-{[(3-methoxybenzyl)sulfonyl]carbamoyl}piperidin-1-yl)-2-methylnicotinate
ethyl 5-cyano-6-(3-{[(2,6-difluorobenzyl)sulfonyl]carbamoyl}azetidin-1-yl)-2-ethylnicotinate
ethyl 5-cyano-2-ethyl-6-(3-{[(4-fluoro-3-methylbenzyl)sulfonyl]carbamoyl}azetidin-1-yl)nicotinate
ethyl 6-(3-{[(2-chloro-4-fluorobenzyl)sulfonyl]carbamoyl}azetidin-1-yl)-5-cyano-2-ethylnicotinate
ethyl 5-cyano-6-(4-{[(5-fluoro-2-methylbenzyl)sulfonyl]carbamoyl}piperidin-1-yl)-2-methylnicotinate
ethyl 5-cyano-2-methyl-6-(4-{[(2,3,6-trifluorobenzyl)sulfonyl]carbamoyl}piperidin-1-yl)nicotinate
ethyl 5-cyano-6-(4-{[(2,4-difluorobenzyl)sulfonyl]carbamoyl}piperidin-1-yl)-2-methylnicotinate
ethyl 5-cyano-6-(4-{[(2,6-difluorobenzyl)sulfonyl]carbamoyl}piperidin-1-yl)-2-ethylnicotinate
ethyl 6-(4-{[(2-chloro-4-fluorobenzyl)sulfonyl]carbamoyl}piperidin-1-yl)-5-cyano-2-ethylnicotinate
ethyl 5-cyano-2-ethyl-6-(4-{[(2,3,6-trifluorobenzyl)sulfonyl]carbamoyl}piperidin-1-yl)nicotinate
ethyl 5-cyano-6-(4-{[(2,4-difluorobenzyl)sulfonyl]carbamoyl}piperidin-1-yl)-2-ethylnicotinate
ethyl 6-(4-{[(4-chloro-2-fluorobenzyl)sulfonyl]carbamoyl}piperidin-1-yl)-5-cyano-2-ethylnicotinate
ethyl 5-cyano-6-(3-{[(2,4-difluorobenzyl)sulfonyl]carbamoyl}azetidin-1-yl)-2-methylnicotinate
ethyl 5-cyano-6-(3-{[(2,4-difluorobenzyl)sulfonyl]carbamoyl}azetidin-1-yl)-2-ethylnicotinate
ethyl 5-cyano-2-ethyl-6-(3-{[(4-fluorobenzyl)sulfonyl]carbamoyl}azetidin-1-yl)nicotinate
ethyl 6-(3-{[(4-chlorobenzyl)sulfonyl]carbamoyl}azetidin-1-yl)-5-cyano-2-ethylnicotinate
ethyl 5-cyano-2-ethyl-6-(4-{[(4-fluorobenzyl)sulfonyl]carbamoyl}piperidin-1-yl)nicotinate
ethyl 6-(4-{[(4-chlorobenzyl)sulfonyl]carbamoyl}piperidin-1-yl)-5-cyano-2-ethylnicotinate
ethyl 5-cyano-6-(3-{[(2-fluorobenzyl)sulfonyl]carbamoyl}azetidin-1-yl)-2-methylnicotinate
ethyl 5-cyano-6-(4-{[(2-fluorobenzyl)sulfonyl]carbamoyl}piperidin-1-yl)-2-methylnicotinate
ethyl 6-{3-[3-(benzyloxy)-3-oxopropyl]-4-[(benzylsulfonyl)carbamoyl]piperidin-1-yl}-5-cyano-2-methylnicotinate
ethyl 6-(3-[3-(benzyloxy)-3-oxopropyl]-4-{[(2,4-difluorobenzyl)sulfonyl]carbamoyl}piperidin-1-yl)-5-cyano-2-methylnicotinate and
3-{4-[(benzylsulfonyl)carbamoyl]-1-[3-cyano-5-(ethoxycarbonyl)-6-methylpyridin-2-yl]piperidin-3-yl}propanoic acid;
or a pharmaceutically acceptable salt thereof

21. A pharmaceutical composition comprising a compound according to claim 1 and a pharmaceutically acceptable adjuvant, diluent, and/or carrier.

22-24. (canceled)

25. A method of treatment of a platelet aggregation disorder comprising administering to a patient suffering from such a disorder a therapeutically effective amount of a compound according to claim 1.
